(19) United States
(12) Patent Application Publication
(10) Pub. No.: US 2024/0279644 A1
(43) Pub. Date: Aug. 22, 2024

(54) TEMPLATE MUTAGENESIS FOR IMPROVED ASSEMBLY OF SEQUENCE READS

(71) Applicant: MGI Tech Co. Ltd, Shenzhen (CN)

(72) Inventors: Brock A. Peters, San Jose, CA (US); Radoje T. Drmanac, Los Altos Hills, CA (US); Amulya Nanisetti, Fremont, CA (US)

(21) Appl. No.: 18/582,080

(22) Filed: Feb. 20, 2024

Related U.S. Application Data
(60) Provisional application No. 63/486,408, filed on Feb. 22, 2023.

(51) Int. Cl. C12N 15/10

(52) U.S. Cl. CPC C12N 15/1065

(57) ABSTRACT

The methods and compositions disclosed herein relate to preparing libraries to sequence long molecules comprising repetitive regions. In some embodiments, this strategy involves substituting selected nucleotides in each molecule to predetermined, different nucleotides at low frequencies in random positions and co-barcoding the molecule in single-tube LFR reactions to generate mutagenized barcoded fragments. Each mutagenized barcoded fragment comprises a barcode, and the barcode comprises a barcode sequence.

### BACKGROUND

This application claims benefit of U.S. Provisional Patent Application No. 63/486,408, filed Feb. 22, 2023, which application is incorporated herein by reference in its entirety for all purposes.

### REFERENCE TO AN ELECTRONIC SEQUENCE LISTING

The content of the electronic sequence listing (106340-1417175-5113-US SL.xml; Size: 17,438 bytes; Date of Creation: Apr. 30, 2024) is herein incorporated by reference in its entirety.

In many organisms, a significant fraction of the genomic DNA is repetitive; over two-thirds of human sequences consist of repetitive elements. The repetitive regions may comprise tandem repeats (i.e., repeats that are directly adjacent to one another in the genome) or interspersed repeats (i.e., repeats dispersed throughout the genome). Some repetitive regions are necessary for maintaining genome structures, such as telomeres or centromere. Some repetitive regions can provide insight into human diseases and genome evolution. Therefore, accurate sequence determination of repetitive regions in the genome is of therapeutic and research interest.

However, sequencing long genomic DNA molecules with highly repetitive nucleotide sequences can be challenging; it is often difficult to decipher whether two identical sequence reads are associated with different positions in the genome or are duplicate sequence reads of the same positions in the genome. There remains a need to identify ways that can accurately determine the sequence information of genomic DNA molecules comprising repetitive regions.

### SUMMARY OF INVENTION

In one aspect, disclosed herein are methods of sequencing a library of target nucleic acid molecules. The method comprises: (a) mutagenizing and co-barcoding the library of target nucleic acid molecules, thereby producing a plurality of mutagenized barcoded fragments for each target nucleic acid molecule, wherein each of the plurality of mutagenized barcoded fragments comprises a barcode, where the barcode comprises a barcode sequence, wherein the plurality of mutagenized barcoded fragments produced from the same target nucleic acid share the same barcode sequence, wherein the mutagenized barcoded fragments produced from different target nucleic acid molecules have different barcode sequences, wherein the mutagenesis converts selected nucleic acid bases to different nucleic acid bases at a rate of 1% to 30%; (b) sequencing the mutagenized barcoded fragments to produce sequence reads, wherein at least some of the sequence reads are significantly overlapping; and (c) assembling the sequence reads to generate an assembled sequence of the target nucleic acid based on the barcode sequence in the sequence reads and mutation patterns.

In another aspect, disclosed herein is a method for sequencing a library of target nucleic acid molecules having repetitive regions, the method comprising co-barcoding the library of target nucleic acid molecules on beads, thereby generating a plurality of barcoded target nucleic acid fragments for each target nucleic acid molecule, wherein for each target nucleic acid molecule, each of the plurality of barcoded target nucleic acid fragments generated for the target nucleic acid comprises a barcode comprising a barcode sequence and a sequence portion of the target nucleic acid molecule, wherein at least some of the plurality of barcoded target nucleic acid fragments have different sequence portions of the target nucleic acid molecule, wherein the barcoded target nucleic acid fragments generated from the same target nucleic acid share identical barcode sequences, and the barcoded target nucleic acid fragments generated from different target nucleic acid molecules have different barcode sequences; wherein for the plurality of the barcoded target nucleic acid fragments generated for the target nucleic acid molecule, denaturing the barcoded target nucleic acid fragments to form single-stranded barcoded fragments, wherein the single-stranded barcoded fragments remain attached to the bead; subjecting the single-stranded barcoded fragments to mutagenesis, which mutates only selected nucleic acid bases at a rate of 1% to 30%, thus forming a group of mutagenized single-stranded barcoded fragments; amplifying the mutagenized single-stranded barcoded fragments to form mutagenized double-stranded barcoded fragments, for each mutagenized double-stranded barcoded fragment, co-barcoding the mutagenized double-stranded barcoded fragment in solution to form a plurality of second double-stranded barcoded fragments, each comprising a copy of the barcode sequence and a sequence portion of the mutagenized double-stranded barcoded fragment, and wherein at least some of the second double-stranded barcoded fragments have different sequence portions of the mutagenized double-stranded barcoded fragment, sequencing the second double-stranded barcoded fragments, thereby producing a plurality of sequence reads, wherein at least some of the plurality of sequence reads are significantly overlapping, and assembling the sequence reads from (ii) to obtain the sequence information of the mutagenized double-stranded barcoded fragments, and assembling the sequence information of the mutagenized double-stranded barcoded fragments to generate the sequence information of the target nucleic acid.

In another aspect, disclosed herein is a method for sequencing a library of target nucleic acid molecules comprising repetitive regions, the method comprising immobilizing the target nucleic acid molecules on beads, wherein each bead is immobilized thereon a barcode comprising a barcode sequence that is unique to the bead; denaturing the target nucleic acid molecules to form single-stranded target nucleic acid molecules, wherein the single-stranded target nucleic acid molecules remain attached to beads; subjecting the single-stranded target nucleic acid molecules to mutagenesis, wherein the mutagenesis mutates only selected nucleic acid bases at a rate of 1% to 30%, thus forming a group of mutagenized single-stranded nucleic acid molecules; performing multiple displacement amplification (MDA) on beads to amplify the mutagenized single-stranded nucleic acid molecules to form amplified mutagenized nucleic acid molecules that remain attached to the beads; co-barcoding the amplified mutagenized nucleic acid molecules on the beads, thereby for each amplified mutagenized nucleic acid molecule, producing a plurality of mutagenized barcoded fragments, each mutagenized barcoded fragment comprising the barcode sequence and a sequence portion of the amplified mutagenized nucleic acid molecule, wherein at least some of the mutagenized barcoded fragment have different sequence portions of the amplified mutagenized nucleic acid molecule; sequencing the mutagenized barcoded fragments to generate sequence reads; and assembling the sequence reads to produce sequence information of the amplified mutagenized nucleic acid molecule, thereby generating the sequence information of the target nucleic acid molecules.

### BRIEF DESCRIPTION OF THE DRAWINGS

The drawings and descriptions thereof illustrate exemplary embodiments of the disclosure. The methods and compositions provided in this disclosure are not limited to the embodiments shown in these drawings.

FIG. 1 shows an exemplary workflow of a method according to Approach I of this disclosure.

FIG. 2A (SEQ ID NO: 10) and 2B (SEQ ID NO: 10 and 11) illustrate exemplary embodiments of the method shown in FIG. 1.

FIG. 3 shows an exemplary workflow of a method according to Approach II of this disclosure.

FIG. 4A (SEQ ID NO: 10 and 11) and 4B (SEQ ID NO: 11 and 12) illustrate exemplary embodiments of the method shown in FIG. 3.

FIG. 5 exemplifies an embodiment of a co-barcoding in solution method. The top panel represents an adaptered double-stranded genomic fragment, which comprises a target sequence with a first end and a second end. The target sequence is flanked by adapter 1 at the 3-prime and adapter 3 at the 5-prime. The first adapter comprises a primer binding site and a barcode sequence, the primer binding site located 3-prime relative to the barcode sequence (not shown).

FIG. 6 exemplifies one exemplary embodiment of a co-barcoding in solution method. Various methods steps are shown including adding barcodes to genomic fragments and amplifying the barcoded genomic fragments.

FIG. 7 exemplifies one embodiment of a method of producing single-stranded DNA circles from the amplified genomic fragments in FIG. 6.

FIG. 8 exemplifies another embodiment of a method of producing single-stranded DNA circles from the amplified genomic fragments in FIG. 6.

FIG. 9 exemplifies another embodiment of a method of producing single-stranded DNA circles from the amplified genomic fragments in FIG. 6.

FIG. 10A exemplifies one embodiment of a method for producing double-stranded adaptered constructs from the single-stranded DNA circles formed as shown in FIG. 7 or FIG. 8.

FIG. 10B exemplifies another embodiment of a method for producing double-stranded adaptered constructs from the single-stranded DNA circles formed as shown in FIG. 7 or FIG. 8.

FIG. 11 exemplifies one embodiment of a method for producing double-stranded adaptered constructs for sequencing according to a linear DNA-based scheme of the co-barcoding in solution method.

### DETAILED DESCRIPTION

### 1. Overview

The methods disclosed herein relate to preparing libraries to determine the sequence of long nucleic acid molecules with repetitive regions. This strategy involves substituting selected nucleotides to different nucleotides in each long nucleic acid molecule at low frequencies in random positions and co-barcoding the long nucleic acid molecule in single-tube LFR reactions to generate mutagenized barcoded fragments. Each mutagenized barcoded fragment comprises a barcode, and the barcode comprises a barcode sequence.

In some embodiments, mutagenesis is performed after co-barcoding of the nucleic acid fragments; see section 5 below entitled “Approach I (Mutagenesis after co-barcoding).” Nucleic acid fragments are first co-barcoded on beads, producing barcoded fragments with lengths of about 1-3, 1-5, 2-5, 3-7, and 3-10 kb. Mutagenesis is then performed on the barcoded fragments attached to the beads.

In some embodiments, mutagenesis is performed before co-barcoding of the nucleic acid fragments; see section 6 below entitled “Approach II (Mutagenesis before co-barcoding).” Mutated nucleic acid fragments are amplified by multiple displacement amplification (MDA) on beads before co-barcoding on the same beads to increase the coverage and continuity of mutagenized contigs.

Mutagenized barcoded nucleic acid fragments are then sequenced, and sequence reads comprising the same barcode sequence are assigned to the same nucleic acid fragment during sequence assembly. Sequence information of the original target nucleic acid fragments (comprising unmutated nucleotides) may be determined by sequence reads generated by sequencing the mutagenized nucleic acid fragments, or by comparing the sequence reads of the mutagenized nucleic acid fragments with the sequence reads of non-mutagenized nucleic acid fragment counterparts. See the section below entitled “Assembling sequence information.”

In scenarios where two or more sequence reads are significantly overlapping, the following procedure is used to determine whether the sequence reads are generated from sequencing the same region in the target nucleic acid or whether they were generated from sequencing different repetitive regions in the target nucleic acids. If the overlapping sequence reads have matching mutation patterns, they are assigned to the same nucleic acid region in the target nucleic acid; if the overlapping sequence reads have distinguishable mutation patterns, they are assigned to different, repetitive regions in the target nucleic acid. See the section below entitled “Assemble sequence information.”

This application incorporates the entire content of WO 2024/022207 by reference.

### 2. Definitions

Components or a reaction in “a single reaction mixture,” means that the reaction occurs in a single mixture without compartmentalization into separate tubes, vessels, aliquots, wells, chambers, or droplets during tagging steps. Components can be added simultaneously or in any order to make the single reaction mixture.

As used herein, “a first end” and “a second end” are used to define the two ends of each nucleic acid molecule in a nest set of nucleic acid molecules. The target sequence near the first ends of the nucleic acid molecules share the same nucleotide sequence and the but differ in nucleotide sequences near the second ends. In a double-stranded DNA molecule, the first end can be either the 5-prime end or the 3-prime end. Similarly, in a double stranded DNA molecule, the second end can be either the 5-prime end or the 3-prime end. Relative to the second end in the same molecule, the first end is closer to the barcode sequence.

As used herein, “unique molecular identifier” (UMI) refers to sequences of nucleotides present in DNA molecules that may be used to distinguish individual DNA molecules from one another. See, e.g., Kivioja, Nature Methods 9, 72-74 (2012). UMIs may be sequenced along with the DNA sequences with which they are associated to identify sequencing reads that are from the same source nucleic acid. The term “UMI” is used herein to refer to both the nucleotide sequence of the UMI and the physical nucleotides, as will be apparent from context. UMIs may be random, pseudo-random, or partially random, or nonrandom nucleotide sequences that are inserted into adapters or otherwise incorporated in source nucleic acid (e.g., DNA) molecules to be sequenced. In some embodiments, each UMI is expected to uniquely identify any given source DNA molecule present in a sample.

As used herein, the term “single-tube LFR” or “stLFR” refers to the process described in, e.g., US patent publication 2014/0323316 and Wang et al., Genome Research, 29: 798-808 (2019), the entire content of each of which is hereby incorporated by reference in its entirety. In stLFR, multiple copies of the same, unique barcode sequence (or “tag”) are associated with individual long nucleic acid fragments. In one embodiment of single-tube LFR, the long nucleic acid fragment is labeled with barcodes at regular intervals. In one embodiment, the barcodes are introduced into the long nucleic acid molecule using one or more enzymes, e.g., transposases, nickases, and ligases. The barcode sequences among different long nucleic acid fragments are different. The barcode sequences among nucleic acid fragments can be conveniently performed in, e.g., a single vessel, without compartmentalization. This process allows analysis of a large number of individual DNA fragments without the need to separate fragments into separate tubes, vessels, aliquots, wells, or droplets during tagging steps.

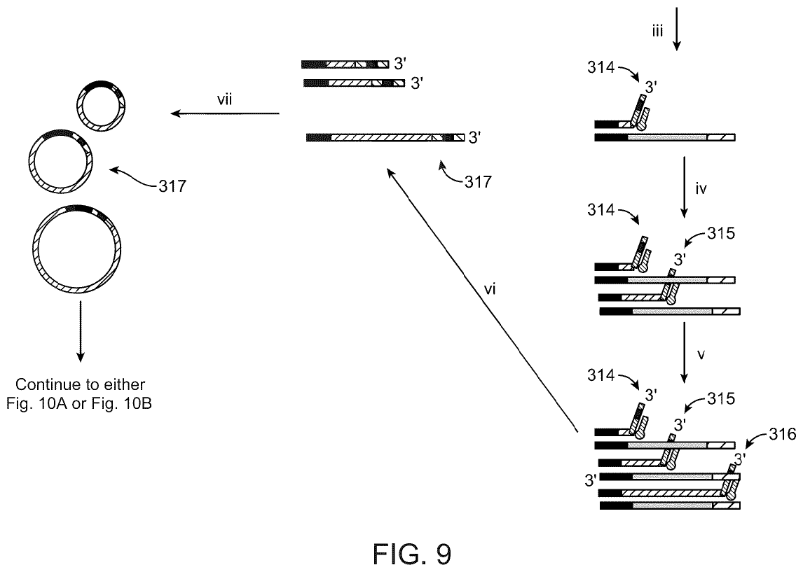

As used herein, a “unique” barcode refers to a nucleotide sequence that is used to identify an individual group of polynucleotides and distinguish it from other groups of polynucleotides among a mixture of groups. For example, a unique barcode for a nested set of nucleic acid constructs means the barcode sequence associated with one nested set is different from the barcode sequence associated with at least 90% of the total nested sets, more often at least 99% of the total nested sets, even more often at least 99.5% of the total nested sets, and most often at least 99.9% of the total nested sets. In some embodiments, a unique barcode is used to identify the position a group of nucleic acid fragments in relation to the genomic DNA from which the group of nucleic acid fragments is derived. A barcode of this type is also referred to as positional barcode in this disclosure. In some cases, different groups of nucleic acid fragments, each group carrying a unique positional barcode, co-exist in one single mixture. See, for example, [316] in FIG. 9. In some cases, different groups of nucleic acid fragments each carrying a unique positional barcode are in separate aliquots and these separate aliquots can then be combined into one mixture.

The term “in solution,” when used to in connection with nucleic acid constructs or polynucleotide complexes used in the methods or compositions disclosed herein, refers to that nucleic acid constructs or polynucleotide complexes are not immobilized on a substrate and can freely move in solution. When used to describe a reaction, as in “a reaction performed in solution” refers to the reaction that occurred between nucleic acids, all of which are in solution.

The term “repetitive region,” refers to a polynucleotide comprising multiple repeats of the same nucleotide sequence.

The term “mutation pattern,” refers to the types of mutated nucleotides (A, T, C, or G) and positions of mutated nucleotide that appear in a sequence read of a mutagenized nucleic acid fragment. Two sequence reads X and Y have matching mutation patterns if X contains the same types of mutated nucleotides that appear in the same positions in Y when X and Y are aligned. Two sequences that have matching mutation patterns may have overlapping sequences or may have identical sequences. Two sequence reads have distinguishable mutation patterns if they do not have matching mutation patterns.

The term “adapter sequence,” refers to a sequence on either strand of an adapter as will be clear from context. That is, “adapter sequence,” can refer to either or both the sequence of an adapter on one strand and the complementary sequence on the second strand. Likewise, the term “barcode sequence,” refers to the sequence of a barcode on one strand or its complementary sequence.

The term “target sequence,” refers to the sequence information of a DNA molecule, e.g., a genomic DNA fragment. Methods and compositions provided herein can be used to determine a target sequence.

The term “sequence portion” refers a portion of the entire sequence or a complement of the sequence of a nucleic acid molecule of interest. Multiple nucleic acid fragments may comprise sequences corresponding to different portions of the same sequence of the nucleic acid molecule.

The term “copy” refers to generating a complementary nucleotide strand of a template by primer extension.

The term “correspond to,” means a DNA sequence has the same or complementary sequence of another DNA sequence.

The term “length suitable for sequencing,” as used herein, refers to that a DNA strand has a length that is equal to the length of a sequence read generated by MPS sequencing. This length may be dictated sequencing methods, but in general the length of a single DNA strand suitable for sequencing falls within a range of 200 bases-1.5 bases, e.g., 300-1000 bases, 300-500 bases, or 400-600 bases or 500-1000 bases, and the length of a DNA duplex suitable for sequencing fall within a range of 200-1.5 base pairs, e.g., 300-1000 base pairs, 300-500 base pairs, or 400-600 base pairs or 500-1000 base pairs.

The term “significant overlapping,” when used to refer to two or more sequence reads, it refers to that the two sequence reads share at least 90%, at least 91%, at least 92%, at least 95%, at least 96%, at least 97%, at least 98%, at least 99% sequence similarity when aligned.

### 3. Methods

### 3.1 Co-Barcoding on Beads

Co-barcoding nucleic acid molecules on beads is a single-tube LFR method of using barcoded beads to introduce barcode sequences at regular intervals in nucleic acid molecules. Each bead is immobilized thereon with a plurality of oligonucleotides. Each oligonucleotide on the bead comprises a barcode comprising a barcode sequence. Oligonucleotides on the same individual bead comprises the same barcode sequence that is unique to the individual bead. The barcode sequence is introduced to the nucleic acid molecules by one or more enzymes, for example, transposases, nickases, and ligases. Co-barcoding on beads are described in Wang et al., Genome Research 29: 798-808 (2019), the entire disclosure of said publications is herein incorporated by reference.

### 3.2 Co-Barcoding in Solution

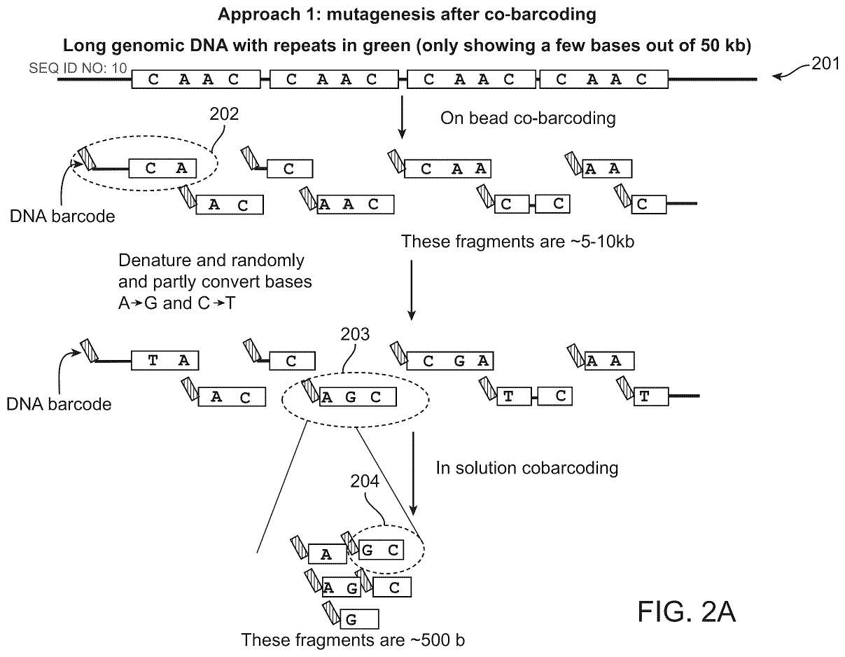

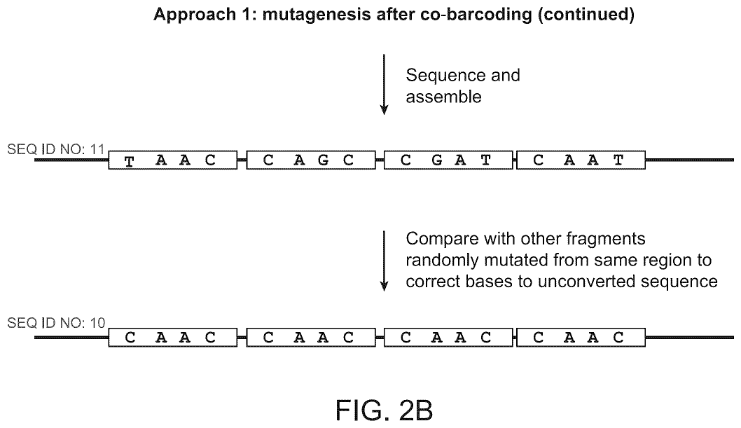

Co-barcoding nucleic acid molecules in solution produces a series of barcoded fragments each comprising a barcode sequence and a sequence portion of the same individual nucleic acid molecule. At least some of the series of barcoded fragments comprise different sequence portions of the individual nucleic acid molecule, and the series of barcoded fragments collectively comprise at least a majority sequence portion of the individual nucleic acid molecule. The term “a majority sequence portion” refers to a sequence portion having a length of at least 90%, at least 95%, at least 98%, or 100% of the entire sequence of the nucleic acid molecule of interest. One exemplary embodiment of co-barcoding nucleic acid molecule in solution is show FIG. 2A, the bottom panel.

Exemplary schemes of co-barcoding fragments in solution are described in section 3.2 and also disclosed in PCT publication WO2024022207, for example in Section 5 entitled “DNA circle-based scheme” and section 6 “linear DNA based scheme.” The entire disclosure of said PCT application is herein incorporated by reference. In one embodiment, the method comprise generating a nested set of nucleic acid constructs for each individual nucleic acid molecule (e.g., individual genomic fragments) and generate a plurality of nested sets for a plurality of nucleic acid molecules. Each nucleic acid construct in each nested set comprises a barcode sequence and a sequence portion of an individual nucleic acid molecule, and nucleic acid constructs within each nested set have different lengths. The nucleic acid constructs in each nested set share a unique barcode sequence. The sequence portions having a first end and second end. Nucleic acid constructs in each nested set share identical nucleotide sequences near the first ends but differ in nucleotide sequences near the second ends.

The nucleic acid constructs are denatured to form single stranded nucleic acid constructs, which are then circularized to form single-stranded circles and each circle comprises the barcode sequence. Upon circularization, the first adapter sequence and the second adapter sequence in each single-stranded nucleic acid construct are joined, which brings the first end and a second end of the target sequence portion into proximity with each other such that a single sequence read can identify the sequence information near both ends.

The circles are then fragmented, and each fragment so produced comprises the barcode sequence that was originally in the DNA circle. Double stranded constructs are then generated from these fragments. Optionally, size selection is performed to select fragments having lengths that are suitable for sequencing.

Various methods can be used to produce the nested set of nucleic acid constructs. Such methods are also described in, for example, PCT publication WO2024022207. In one embodiment of such methods, the individual nucleic acid molecule is ligated to an adaptor comprising the barcode sequence and then amplified to produce amplified nucleic acid fragments. The nucleic acid fragments can be treated with nicking agents to introduce nicks at random positions. This process results amplified nucleic acid fragments being truncated, forming the nested set of nucleic acid constructs described above.

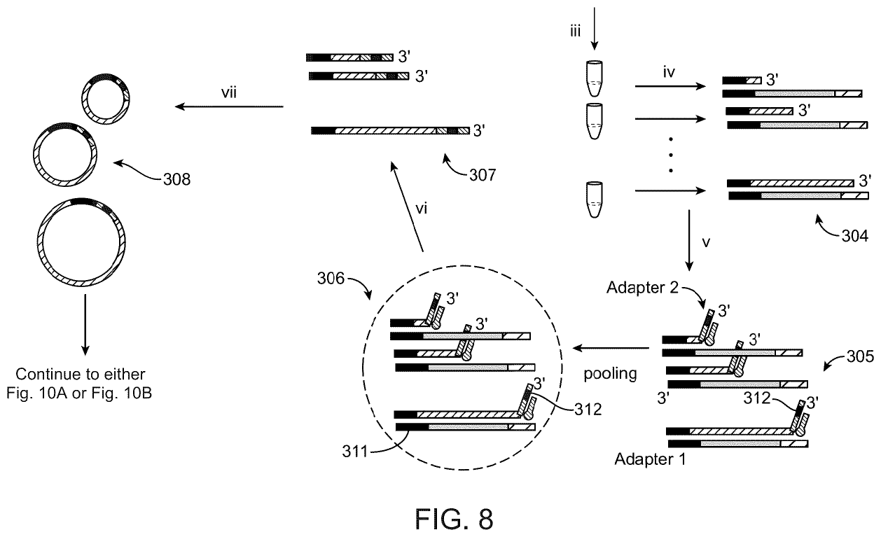

In some embodiments, the series of barcoded fragments produced from co-barcoding of an individual nucleic acid molecule in solution further comprise positional barcodes. Each positional barcode comprises a positional barcode sequence, and at least two positional barcode sequences in the series of barcoded fragments are different. The different positional barcode sequences denote different positions of the barcode fragments relative to the individual nucleic acid molecule from which barcode fragments are derived. Co-barcoding in solution methods are also described below and shown in FIGS. 8 and 9.

### 3.2.1. DNA Circle-Based Scheme

In some approaches, the co-barcoding in solution method use a DNA circle-based approach. The methods comprise circularizing the nucleic acid constructs in each nested set so that the two ends in each nucleic acid construct are joined together. See FIG. 5. Each nucleic acid construct comprises a target sequence flanked by a first adapter (“Adapter 1”) sequence and a third adapter (“Adapter 3”) sequence. Circularization of the nucleic acid constructs allows the sequences near both ends to be included in a single sequence read. The target sequence portions from different nucleic acid constructs in the same nested set can be assembled to generate sequence information that corresponds to the entire target sequence in the genomic fragment. The scheme is further illustrated below in detail.

### Ligating Adapters to Both Ends of Genomic Fragments and Amplifying Genomic Fragments

One exemplary approach of adding adapters to both ends of genomic fragments [201] is illustrated in FIG. 6. Step (i) shows ligating double-stranded genomic fragments to two double-stranded adapters at both ends to produce adaptered genomic fragments [202]. Step (ii) shows amplifying the adaptered double-stranded genomic fragments. Amplification can be performed using primers hybridizing primers to the first and third adapter sequences (not shown). This step results in amplified genomic fragments [203] with blunt ends.

### Producing Nested Sets of Single-Stranded Nucleic Acid Constructs Comprising Target Sequences and Circularization

In the circularization approach, the amplified genomic fragments are processed to produce nested sets of single-stranded nucleic acid constructs [303] (FIG. 7), and the single-stranded nucleic acid constructs are circularized. Each single-stranded DNA construct comprises a target sequence portion that has a first end and a second end. The target sequence portion is flanked by the first adapter sequence abutting the first end. A second adapter sequence (Adaptor 2) is inserted adjacent to the second end. In some approaches, the first adapter sequence is at the 5-prime of the single-stranded DNA construct, and the second adapter sequence is at the 3-prime. The first adapter sequence comprises a barcode sequence that is unique to individual nested sets, i.e., single-stranded DNA constructs within the same nested set share the same barcode sequence and single-stranded DNA constructs from different nested sets have different barcode sequences. In some cases, the first end of the single-stranded DNA construct is closer to the barcode sequence than the second end is.

Various approaches can be used to produce single-stranded nucleic acid constructs from the amplified genomic fragments. In some approaches, a nested set of single-stranded DNA constructs are generated by contacting amplified genomic fragments with nicking agents to introduce nicks in the target sequence. Then second adapters are ligated at the nicks via branch ligation. One example of such an approach is illustrated in FIG. 7. Step (iii) of FIG. 7 shows contacting the amplified genomic fragments (generated from (ii) from FIG. 6) with a nicking agent to produce nicks at random positions in the target sequences in the amplified genomic fragments. Each amplified genomic fragment may be nicked one or more times, and the nicks produced in the fragment can be extended by using one or more exonucleases to form gaps. This process results in one or more fragments, but only one of the fragments contains the first adapter sequence (comprising the barcode sequence [311]). A number of parameters can affect the length of the nucleic acid fragments separated by the nicks and/or gaps. Typically, the higher the concentration of the nicking agent, the longer treatment time by the nicking agents, the shorter the length the fragments. By adjusting one or more of these parameters, the length of the fragments can be controlled within a desired range. In some embodiments, the average length of the nucleic acid fragments resulted from the nicking is between 200 and 10000 nucleotides, e.g., 200-500 nucleotides or 400-1000 nucleotides or 1000-10000 nucleotides. Step (iv) shows ligating a second adapter comprising a second adapter sequence at the nicks via branch ligation to form ligated products [302], each of some of the ligated products comprises a first adapter sequence and a second adapter sequence. Step (v) shows denaturing the ligated products to form single-stranded nucleic acid constructs [303], each of some ligated products comprising the first adapter sequence and the second adapter sequence. The population of single-stranded nucleic acid constructs represent a nested set of constructs comprising target sequence.

In some other approaches, generation of a nested set of single-stranded DNA constructs involves annealing a primer to the primer binding sequence in the first adapter that has been ligated to the genomic fragment and extending the primer to produce a primer extension product. One example of such an approach is shown in FIG. 8. Step (iii) of FIG. 8 shows distributing the amplified, adaptered double-stranded genomic fragments [203] as shown in FIG. 6 into a plurality of aliquots. Step (iv) shows denaturing the amplification products in each aliquot to form single-stranded molecules [304] and then hybridizing a primer to the primer-binding sequence. Said primer is extended in the presence of polymerase and dNTPs, and the extension is controlled such that the extension products in different aliquots have different lengths, thereby forming a nested set of extension products. The extension products each has a first end (where the primer starts) and a second end (where the extension ends), and the extension products share the same sequence near the first ends and have different sequences near the second ends. Step (v) shows adding second adapters to the second ends by branch ligation. These second adapters comprise positional barcode sequences [312] that are unique to individual aliquots. As a result, single-stranded nucleic acid constructs [305] formed as the result of the branch ligations in different aliquots comprise different positional barcode sequences. The single-stranded nucleic acid constructs in the same aliquot share the same positional barcode sequence. The aliquots are then combined into one single mixture [306]. The molecules shown inside the dashed oval are in one single mixture. Subsequent steps are also all performed in one single mixture. Step (vi) shows denaturing the product in the single mixture to form adaptered fragments [307].

In some other approaches, generating a nested set of single-stranded DNA constructs involves ligating adapters via branch ligation after each specified period of time, where the adapters comprise positional barcode sequences. One exemplary approach is shown in FIG. 9. Unlike the approach illustrated in FIG. 8, the reactions in FIG. 9 are performed in a single tube throughout the entire procedure-no aliquoting is needed. Step iii shows ligating a second adapter to the extended primers after the primer is extended for a first time period, resulting in a reaction mixture of fragments ligated with the second adapter [314]. Step iv shows ligating an additional adapter to the extended primers (in the same reaction mixture) after the primer is extended for an additional time period, resulting in a mixture of (a) fragments ligated with the second adapter [314] and (b) fragments ligated with a first additional adapter [315]; step v shows adding a second additional adapter after the primer is extended for yet another period of time, resulting a mixture of fragments ligated with the second, first additional, and second additional adapters [314], [315], and [316], and so on. The process of extending the primer for an additional period of time and then adding adapters with unique positional barcodes can be repeated for 3-50 rounds, e.g., 5-20 rounds, 10-40 rounds, 15-30 rounds, 20-40 rounds, or 10-20 rounds. Each of the second, first additional, second additional adapter, and further additional adapters comprises a unique positional barcode sequence, and ligation of each of these adapters to the extended primer is abranch ligation.

The molar amount of each of the adapters used in the branch ligation (as illustrated in steps (iii)-(v) of FIG. 9 is a small percentage of the total molar amount of the amplified genomic fragments, such that only a fraction of the extended primers that are available for branch ligation in each round are ligated with the adapter. In some embodiments, the molar amount of the adapter used is 1-20%, e.g., 1-10%, or 2-15% of the total molar amount of the amplified genomic fragments. In some embodiments, the amounts of the adapters (e.g., the second adapter, the first additional adapter, the second additional adapter) used in different rounds are same. In some embodiments, the amounts of the adapters used in different rounds are different. Step (vi) of FIG. 9 shows denaturing the reaction mixture [316] to produce single-stranded, adaptered fragments [317]. The single-stranded fragments are then circularized as described below.

### Circularization

The single-stranded nucleic acid constructs are then circularized to form single-stranded circles. Methods for circularization of single-stranded nucleic acids are well known. At least some of these single-stranded DNA fragments comprise the barcode sequence and a target sequence portion. In each nested set, target sequence portions share the same nucleotide sequence near the first ends but have different nucleotide sequences near the second ends. Upon circularization, the first adapter sequence and the second adapter sequence in each single-stranded nucleic acid construct are joined, which brings the first end and a second end of the target sequence portion into proximity with each other such that a single sequence read can identify the sequence information near both ends. Exemplary approaches are illustrated in FIG. 7, step (vi) [308] and FIG. 8, step (vii) [306].

Producing Linear Double-Stranded Adaptered Constructs from Single-Stranded Circles

Various approaches can be used to producel linear, double-stranded, adaptered constructs from single-stranded DNA circles (for example those generated using methods in FIG. 7 or FIG. 8 or FIG. 9. Some of these approaches involves random fragmentation of the single-stranded circles to produce fragments and generating double stranded, adaptered constructs from these fragments. DNA circles can be fragmented using methods well known in the art, for example, sonification, to produce a plurality of single-stranded DNA fragments. Each single-stranded DNA fragment comprises the barcode sequence that was in the DNA circle. Optionally, size selection is performed to select fragments having lengths that are suitable for sequencing.

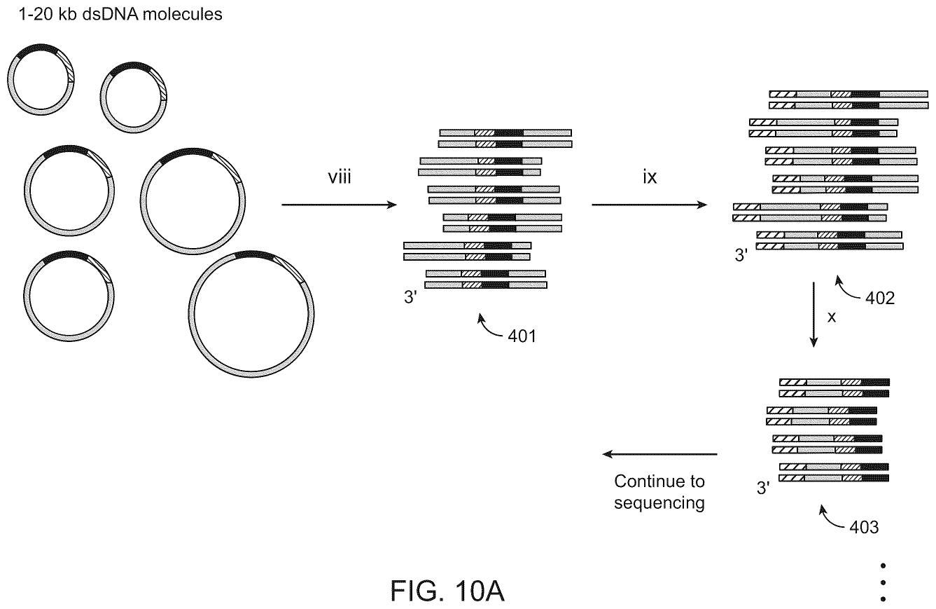

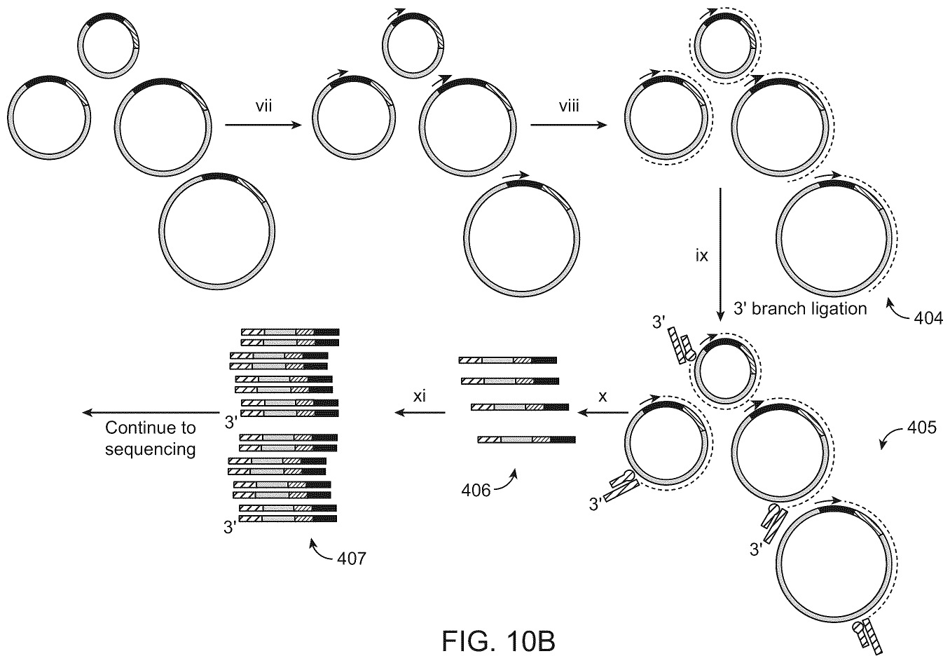

One example of such approaches is illustrated in FIG. 10A. Complementary strands are synthesized using the single-stranded DNA fragments as templates, resulting in formation of a plurality of double-stranded fragments [401]. Step (viii) shows ligating adapters to both ends of the double-stranded fragments to produce adaptered double-stranded constructs [402]. Step (ix) shows performing size selection to generate nucleic acid constructs having lengths suitable for sequencing [403].

In some other approaches, linear, adaptered, double-stranded constructs are generated by extending a primer hybridized to the circle under extension-controlling conditions to produce extended primers having lengths suitable for sequencing. One illustrative example is shown in FIG. 10B, steps (vii) and (viii). Typically, the controlled extension does not result in copying the entire template sequence; rather, the extended primers remain hybridized to the circles [404] with 3-prime recessed ends that are ready for branch ligation. In some cases, the extended primers have a length within the range of 300-1000 bases, e.g., 300-500 base or 400-600 base, to achieve more efficient sequencing. Any short artifact products can be removed through exonuclease treatment or purification. Thus, size selection is not necessary with this approach and all extension products can be used in generating the sequence reads.

A second adapter is then ligated to the recessed 3-prime ends of the extended primers via branch ligation to form adaptered extended primers, each having a second adapter sequence on one end and the primer binding sequence and the barcode sequence on the other end. 10B, step (ix) [405]. The adaptered extended primers [406] are collected (FIG. 10B, steps (x)), and primer extension is performed using the adaptered extended primers as templates to produce the complementary strands and then form double-stranded DNA fragments [407]. FIG. 10B, steps (xi). The double-stranded DNA fragments can then be amplified and sequenced.

A nested set of linear double stranded fragments for each genomic fragment to be sequenced can be generated using the DNA circle-based scheme as described above. Each of the double-stranded DNA fragments in the nested set comprises different target sequence portions of the genomic fragment, and these different target sequence portions together can be assembled to decipher the sequence of the original long DNA molecule. See the section above entitled “assemble sequence information.”

### 3.2.2. Linear DNA-Based Scheme

In some approaches, co-barcoding in solution uses a linear DNA-based approach as further disclosed below. This process does not generate DNA circles.

### Adding Adapters to Both Ends of Economic Fragments and Amplify Genomic Fragments

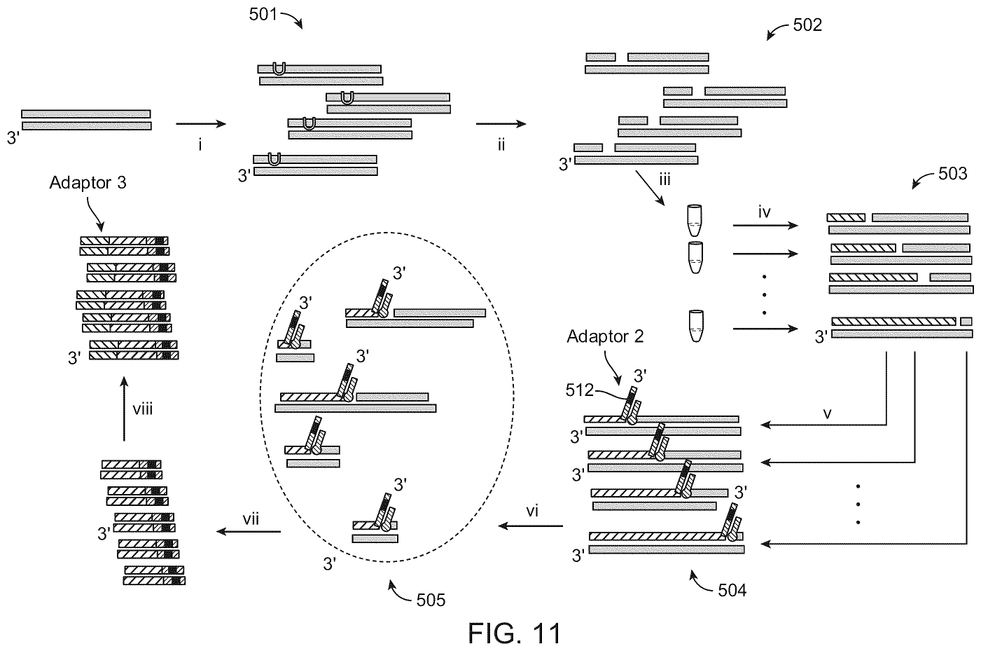

First, adapters are added to both ends of genomic fragments as illustrated in FIG. 6 and also described in section 3.2.1 above. In some approaches, the genomic fragments are amplified similar to section 3.2.1 above except that the amplification is carried out by a polymerase in a reaction mixture containing uracils or using primers comprising uracils, thereby producing amplified nucleic acid fragments incorporating uracils in the reaction mixture. One example is shown in FIG. 11, step (i), where the uracils are part of the amplification primers (not shown) and are incorporated into the amplified genomic fragments during amplification.

### Creating Nicks

Next, nicks are introduced into amplified genomic fragments by enzymatic digestion. In some approaches, the amplification is in the presence of uracils as described above, and nicks can be introduced to the amplified genomic fragments containing the uracils by contacting them with a uracil-DNA glycosylase. The uracil glycosylase can remove the uracils to form abasic sites. An enzyme (e.g., APE1 or EndoIV) is also added to the reaction to remove the sugar groups from abasic sites. This treatment of the uracil-containing genomic fragments using the enzymes as described above results in nicks the extension products in the region containing uracil bases, each nick flanked by a 5-prime exposed terminus and a 3-prime exposed terminus.

Preferably, uracils are spiked to the amplification reaction after the extension of the amplification primer has passed the barcode region but before reaching an extension length that is approximately the size of the desired read length, also referred to as a length that is suitable for sequencing. The length that is suitable for sequencing may be in a range between 25-1000 bases, depending on the read length dictated by the sequencing methods. In some approaches, this is accomplished by spiking uracils into the reaction mixture after the extension has already been initiated, i.e., when all other components required for amplification have already been added to the reaction mixture. In some approaches, uracils are spiked to the reaction mixture roughly 10 seconds to 10 minutes after the initiation of the extension.

In other approaches, primers used to amplify the genomic fragments comprise the uracils, which are incorporated into the amplified genomic fragments [501]. In some embodiments, the forward primer comprise one or more uracils. In some embodiments, each forward primer comprises a single uracil such that one nick is generated in each double-stranded nucleic acid fragment [502] (after performing enzymatic treatment to remove the uracils, as described above).

### Aliquoting

The reaction mixture is then distributed into a plurality of aliquots. See FIG. 11, step (iii).

### Nick Translation to Produce a Nested Set of Nucleic Acid Constructs

Next, nick translation is performed with a DNA polymerase with a 5′→3′ exonuclease activity in the aliquots to synthesize DNA strands with newly formed ends (second ends). Nonlimiting examples of DNA polymerases include DNA Pol I, Taq, Bst full length, and Pfu DNA polymerase. The ends that are opposite to the second ends are the first ends. The extension is controlled such that the DNA strands synthesized in different aliquots have different lengths. Each synthesized DNA strand comprises a first end and a second end, and the DNA strands in different aliquots share the same sequence near the first ends and have different sequences near the second ends [503]. Each of the DNA strands synthesized comprises a target sequence portion with a first end and a second end, the second end being the end formed by the nick translation and the first end being the end opposite the second end. The DNA strands in different aliquots share the same sequence near the first ends and have different sequences near the second ends. One illustrative example is shown in FIG. 11, step (iv).

### Branch Ligation

Branch ligation, also referred to as “3-prime ligation” or “3-prime branch ligation,” relies on a property of T4 ligase, ligates a double-stranded DNA adapter to a 3-prime end of DNA in an interval or gap. See, Wang et al., DNA Research, 2019 Feb. 1 16(1):45-53, the entire disclosure is herein incorporated by reference. Branch ligation is efficient in ligating adapters because it does not require degenerate single-stranded bases on the end of the adapter to hybridize in the gap.

Adapters suitable for use in the branch ligation typically comprise: (i) a double-stranded blunt end comprising a 5-prime terminus of one strand and a 3-prime terminus of the complementary strand (ii) a single-stranded region comprising a barcode sequence. The double-stranded blunt end provides a 5-prime phosphate which can be ligated to the 3-prime of the target nucleic acid fragments via 3-prime branch ligation. In some embodiments, the double-stranded blunt end provides a 3-prime that is blocked from ligation by a dideoxynucleotide, 3′ phosphate group, 3′ overhang or the like. 3-prime branch ligation involves the covalent joining of the 5-prime phosphate from a blunt-end adapter (donor DNA) to the 3-prime hydroxyl end of a duplex DNA acceptor at 3-prime recessed strands, gaps, or intervals. In contrast to conventional DNA ligation, 3-prime branch ligation does not require complementary base pairing. 3-prime branch ligation is described in Wang et al., DNA Res. 26 (1): 45-53, doi:10.1093/dnares/dsy037; PCT Pub. No. WO 2019/217452; US Pat. Pub. US2018/0044668 and International Application WO 2016/037418, US Pat. Pub. 2018/0044667, all incorporated by reference for all purposes.

### Branch Ligation in Individual Aliquots

Adapters (second adapters) are added to the aliquots after the completion of the nick translation reactions. These second adapters are ligated to the second ends of the newly synthesized DNA strands. Each second adapter is partially double stranded and comprises a first adapter oligonucleotide and a second adapter oligonucleotide. The first and second adapter oligonucleotides are complementary and hybridized to each other. During branch ligation, the 5-prime end of the first adapter oligonucleotide is joined to the 3-terminus of the second end of a DNA strand synthesized via nick translation as described above [504], step (v).

In some approaches, the second adapter comprises a positional barcode that is unique to the aliquot. In some approaches, the aliquots now comprising the unique positional barcodes are then combined into one single reaction mixture [505].

In some approaches, the second adapter further comprises an anchoring component for separating fragments ligated to second adapters from those that are not ligated to the second adapters. In some approaches, the anchoring component allows the adaptered fragments to be captured by solid supports and the captured adaptered fragments can then be isolated from other reagents in solution. In some approaches, the anchoring component can be a biotin, and the solid support is coated with streptavidin. In some approaches, the anchoring component is an oligonucleotide in the second adapter and the solid support is a magnetic bead with oligonucleotides immobilized thereon.

The synthesized DNA strands ligated with the second adapters from different aliquots from (v) are then combiend to form in a single mixture [505]. FIG. 11, step (vi). The molecules shown inside the dashed oval are in one single mixture. Subsequent steps are all performed in one single mixture.

### Extending the Second Adapter Oligonucleotide of the Second Adapter to Form Double-Stranded Fragments

The branch ligation results in the first adapter oligonucleotide joined to the nucleic acid constructs and the second adapter oligonucleotide not joined but remain hybridized to the now joined first adapter oligonucleotide. A primer is then hybridized to the first adapter oligonucleotide, and the hybridized primer is extended are to generate double-stranded fragments. In some approaches the double-stranded fragments so produced have blunt ends. In some approaches, the double-stranded fragments so produced comprise positional barcodes that are unique to individual aliquots. One illustrative example of this embodiment is shown in FIG. 11, step (vii).

### Producing Linear Double-Stranded Adaptered Constructs

Optionally, the double-stranded DNA molecules having the lengths that are suitable for sequencing are selected. In some approaches, the double-stranded fragments having lengths within a range from 200 bp-1.5 kb, e.g., from 200-1200 bp, from 200-1000 bp, from 400-1200 bp, from 400-1000 bp, from 500-1500 bp, from 500-1200 bp, or from 500-1000 bp, are selected. In some approaches. The selected double-stranded fragments are ligated to adapters (“third adapters”) via e.g., blunt-end ligation, thereby producing double-stranded adaptered constructs. See, FIG. 11, Step (viii). The double-stranded adaptered constructs can then be sequenced as disclosed herein.

The sequences near the positional barcodes in the double stranded fragments in individual aliquots can be determined by sequencing, and sequence reads corresponding to different target sequence portions in individual nucleic acid constructs are assembled to generate the sequence information for the entire target sequence.

### Mutagenesis

In some embodiments, the mutagenesis results in deamination of one of the nucleotides. In one or more embodiments, the mutagenesis is by cytosine deamination, which results in C→T mutation. In one or more embodiments, the mutagenesis is by adenine deamination, which results in A→G mutation.

Mutations can be introduced by treating the nucleic acid fragments with various chemical compounds or enzymes. In one embodiment, the fragments are treated with sodium nitrate. Methods of using sodium nitrate to introduce random mutations is described in Mahdavi-Amiri et al., Chem. Sci. (2021) Jan. 14; 12 (2): 606-612, the entire disclosure of which is incorporated by reference. In another embodiment, the fragments are treated with bisulfite, and methods of using bisulfite to introduce random mutations is described in Li et al., Nucleic. Acids Res. (2022) Oct. 14; 50 (18): e103, available at ncbi.nlm.nih.gov/pmc/articles/PMC9561374/. The entire content of said publications is herein incorporated by reference. In yet another embodiment, APOBEC, a apolipoprotein B mRNA editing enzyme, catalytic polypeptide, can be used to introduce C→U mutations in mRNA fragments.

The frequency of mutagenesis introduced by the method described above is low, for example about 1% to 30%, from 1% to 20%, from 2% to 15%, from 3% to 10%, from 2% to 10%, from 1% to 3%, or from 2% to 5%. The mutagenesis frequency can be controlled by methods that are well known, for example, controlling concentrations of the chemical compound or enzyme, reaction temperatures and the like. In some embodiments, 0.1M to 10M, 0.5M to 2M, e.g., 1M sodium nitrate in the reaction mixture is used to deaminate Adenine (A) to Guanine (G) and deaminate cytosine (C) to thymidine (T) in nucleic acid fragments.

Advantageously, the nucleic acid fragments remain attached to the beads before, during, and after the mutagenesis. The beads, with mutagenized nucleic acid fragments attached thereon, are collected. In some cases the beads are magnetic and can be conveniently collected using a magnetic plate. In some embodiments the collected beads are washed before the downstream processing steps are performed.

### Amplification

Amplification methods used in the disclosure include but not limited to: multiple displacement amplification (MDA), polymerase chain reaction (PCR), ligation chain reaction (sometimes referred to as oligonucleotide ligase amplification OLA), cycling probe technology (CPT), strand displacement assay (SDA), transcription mediated amplification (TMA), nucleic acid sequence-based amplification (NASBA), rolling circle amplification (RCR) (for circularized fragments), and invasive cleavage technology. Amplification can be performed after fragmenting or before or after any step outlined herein.

In some approaches, amplification is performed on barcoded genomic fragments by extending primers annealed to the adapter sequences. In some approaches, the genomic fragments having different target sequences are ligated to adapters at both ends, and the genomic fragments are then amplified using the primers hybridized to the adapters at both ends. In some approaches one of the adapters comprises a barcode. In some embodiments, the adapters are ligated to the genomic fragments by branch ligation.

In some approaches, the amplification is performed using target-specific primers, i.e., primers that hybridize to target sequence in the genomic DNA. In some approaches, the target-specific primers containing a common adapter tag with a random barcode to amplify specific regions.

In some approaches, the amplification can be a multi-plex PCR, i.e., using multiple primer pairs targeting different target sequences in the genomic DNA. In some approaches, the amplification is a multiplex PCR in which 2-1000 of different target regions are amplified using target-specific primers in one reaction, such that the reaction mixture comprises amplified genomic fragments having different target sequences.

In some approaches, genomic fragments or barcoded nucleic acid fragments can be amplified using rolling circle amplification (RCR). Genomic fragments are first denatured into single-stranded nucleic acid molecules. A splint oligo is added and hybridized to the adapter sequences flanking the target sequences, and the single-stranded nucleic acids are then circularized in the presence of a ligase (e.g., T4 or Taq ligase). The DNA polymerase used for RCR can be any DNA polymerase that has strand-displacement activity, e.g., Phi29, Bst DNA polymerase, Klenow fragment of DNA polymerase I, and Deep-VentR NDA polymerase (NEB #MO258). These DNA polymerases are known to have different strengths of strand-displacement activity. It is within the ability of one of ordinary skill in the art to select one or more suitable DNA polymerase used for the invention.

### Sequencing

Libraries of barcoded fragments can be sequenced using methods known in the art, including for example without limitation, polymerase-based sequencing-by-synthesis (e.g., HiSeq 2500 system, Illumina, San Diego, CA), ligation-based sequencing (e.g., SOLiD 5500, Life Technologies Corporation, Carlsbad, CA), ion semiconductor sequencing (e.g., Ion PGM or Ion Proton sequencers, Life Technologies Corporation, Carlsbad, CA), zero-mode waveguides (e.g., PacBio RS sequencer, Pacific Biosciences, Menlo Park, CA), nanopore sequencing (e.g., Oxford Nanopore Technologies Ltd., Oxford, United Kingdom), pyrosequencing (e.g., 454 Life Sciences, Branford, CT), or other sequencing technologies. Some of these sequencing technologies are short-read technologies, but others produce longer reads, (e.g., the GS FLX+(454 Life Sciences; up to 1000 bp), PacBio RS (Pacific Biosciences; approximately 1000 bp) and nanopore sequencing (Oxford Nanopore Technologies Ltd.; 100 kb). For haplotype phasing, longer reads are advantageous and require much less computation, although they tend to have a higher error rate and errors in such long reads may need to be identified and corrected according to methods set forth herein before haplotype phasing.

In some approaches, sequencing is performed using combinatorial probe-anchor ligation (cPAL) as described in, for example, US 20140051588, U.S. 20130124100, both of which are incorporated herein by reference in their entirety for all purposes.

In some approaches, sequencing is performed using DNBseq sequencers. The barcoded fragments or amplified products thereof are denatured to produce single-stranded molecules. These circles are then used to make DNA nanoballs (DNBs) for DNBseq sequencers.

In some approaches, the barcoded fragments or amplified products thereof are sequenced on Illumina or other systems that do not require circularization.

In some approaches, the sequencing is a paired-end sequencing comprising sequencing from either terminus of the same DNA fragment. In some approaches, first read reads are produced by extending a sequencing primer annealed to the adapter sequence that is closer to the first end of the target sequence fragment than the second end (“first read sequencing”), and second sequencing reads are produced by extending a sequencing primer annealed the adapter sequence that is closer the second end of the target sequence fragment than the first end (“second read sequencing”). In some approaches, the first read sequencing will produce the barcode sequence. The second read sequencing will produce overlapping reads to substantially or completely cover molecules up to 500 bp or 700 bp or 1000 bp in length. These overlapping sequencing reads would be clustered based on the barcode sequence determined by the first read sequencing in a de novo assembly.

In some approaches, the sequencing is a single-end sequencing, which produces the sequence information of the genomic fragment.

### Assembling Sequence Information

Generating the sequence of the original long nucleic acid molecules comprises (1) clustering the sequence reads based on the shared barcode sequence (2) reverting the mutated nucleotides to unmutated nucleotides and (3) determining the sequence of a region comprising multiple repeats. In some embodiments, step (1) is performed before step (2). In some embodiments, step (2) is performed before step (1).

For procedure (1), sequence reads sharing the same barcode are clustered and assigned to the same individual nucleic acid molecule.

For procedure (2), reverting mutated nucleotides to unmutated nucleotides refers to determining the identity of the unmutated nucleotides in the same positions of the sequence reads had the mutagenesis described above not performed. This procedure sometimes is also referred to as base correction in this application. Reverting mutated nucleotides to unmutated nucleotides can be performed in different ways. In some embodiments, reverting mutated nucleotides to unmutated nucleotides comprises comparing overlapping sequence reads with mutated bases. As discussed above, mutagenesis used in the methods is typically controlled and occurs at a low frequency, resulting a majority of nucleotides in the same position of the sequence reads remain unmutated. In genomic regions of low complexity or comprising many repeats, for example, repeats in tandem, these mutated bases are helpful to determine the order and overlap of reads: the mutated bases create uniqueness between otherwise repetitive sequences. This uniqueness allows identification of overlapping reads that share the same mutated bases, which can be used to assemble the sequence reads. Thus, by comparing overlapping sequence reads cover a given region, the original (unmutated) base at each position can be determined. In some embodiments, reverting mutated nucleotides to unmutated nucleotides can be performed by comparing sequence reads generated above with control sequence reads (aka., unmutated sequence reads). These unmutated sequence reads can be obtained in different ways. In some embodiments, the unmutated sequence reads can be generated from sequencing the barcoded fragments generated from subjecting the same individual nucleic acid molecules to the same process above except for mutagenesis. In some embodiments, the unmutated sequence reads are obtained from a standard sequencing library.

For procedure (3), in scenarios where sequence reads have significant overlapping, overlapping sequence reads having distinguishable mutation patterns are assigned to different, repetitive regions of the individual nucleic acid molecule, and overlapping sequence reads having matching mutation patterns are assigned to the same region of the individual nucleic acid molecule. The sequence of the original long nucleic acid molecule can be assembled from the unmutated sequence reads using methods well known in the art. For example, in some cases, the overlapping K mers of the sequence reads are identified, and two sequence reads are connected into one continuous sequence if the nucleotides at the edge of the first read overlap the edge of the second read (and so on). The process is repeated to add additional sequence reads at either direction to extend the assembled contig, generating the sequence information for the original nucleic acid molecule.

### 4. Compositions

### 4.1 Samples

Samples containing target nucleic acids can be obtained from any suitable source. For example, the sample can be obtained or provided from any organism of interest. Such organisms include, for example, plants; animals (e.g., mammals, including humans and non-human primates); or pathogens, such as bacteria and viruses. In some cases, the sample can be or can be obtained from, cells, tissue, or polynucleotides of a population of such organisms of interest. As another example, the sample can be a microbiome or microbiota. Optionally, the sample is an environmental sample, such as a sample of water, air, or soil.

Samples from an organism of interest, or a population of such organisms of interest, can include, but are not limited to, samples of bodily fluids (including, but not limited to, blood, urine, serum, lymph, saliva, anal and vaginal secretions, perspiration and semen); cells; tissue; biopsies, research samples (e.g., products of nucleic acid amplification reactions, such as PCR amplification reactions); purified samples, such as purified genomic DNA; RNA preparations; and raw samples (bacteria, virus, genomic DNA, etc.). Methods of obtaining target polynucleotides (e.g., genomic DNA) from organisms are well known in the art.

### 4.2 Target Nucleic Acid

As used herein, the term “target nucleic acid” (or polynucleotide) or “nucleic acid of interest” refers to any nucleic acid (or polynucleotide) suitable for processing and sequencing by the methods described herein. In some approaches, the target nucleic acid is a genomic fragment, generated by fragmenting genomic DNA extracted from a sample. It is noted that while genomic fragments are used for illustration of the methods and compositions disclosed herein, sequencing libraries can also be prepared using these methods and compositions to sequence any target nucleic acid or fragments thereof, including those that contain modifications of the nucleotides, e.g., nucleotide analogs.

The nucleic acid may be single-stranded or double-stranded and may include DNA, RNA, or other known nucleic acids. The target nucleic acids may be those of any organism, including, but not limited, to viruses, bacteria, yeast, plants, fish, reptiles, amphibians, birds, and mammals (including, without limitation, mice, rats, dogs, cats, goats, sheep, cattle, horses, pigs, rabbits, monkeys and other non-human primates, and humans). A target nucleic acid may be obtained from an individual or from multiple individuals (i.e., a population). A sample from which the nucleic acid is obtained may contain nucleic acids from a mixture of cells or even organisms, such as: a human saliva sample that includes human cells and bacterial cells; a mouse xenograft that includes mouse cells and cells from a transplanted human tumor; etc. Target nucleic acids may be unamplified or they may be amplified by any suitable nucleic acid amplification method known in the art. Target nucleic acids may be purified according to methods known in the art to remove cellular and subcellular contaminants (lipids, proteins, carbohydrates, nucleic acids other than those to be sequenced, etc.), or they may be unpurified, i.e., include at least some cellular and subcellular contaminants, including without limitation intact cells that are disrupted to release their nucleic acids for processing and sequencing. Target nucleic acids can be obtained from any suitable sample using methods known in the art. Such samples include but are not limited to biosamples such as tissues, isolated cells or cell cultures, bodily fluids (including, but not limited to, blood, urine, serum, lymph, saliva, anal and vaginal secretions, perspiration and semen); and environmental samples, such as air, agricultural, water and soil samples, etc.

Target nucleic acids may be genomic DNA (e.g., from a single individual), cDNA, and/or may be complex nucleic acids, including nucleic acids from multiple individuals or genomes. Examples of complex nucleic acids include a microbiome, circulating fetal cells in the bloodstream of an expecting mother (see, e.g., Kavanagh et al., J. Chromatol. B 878: 1905-1911, 2010), circulating tumor cells (CTC) from the bloodstream of a cancer patient. In one embodiment, such a complex nucleic acid has a complete sequence comprising at least one gigabase (Gb) (a diploid human genome comprises approximately 6 Gb of sequence).

In some cases, target nucleic acids are genomic fragments. In some approaches the genomic fragments are longer than 10 kb, e.g., 10-100 kb, 10-500 kb, 20-300 kb, 50-200 kb, 100-400 kb, or longer than 500 kb. In some cases, target nucleic acids are 5,000 to 100,000 Kb. In some approaches, the target nucleic acids are 500 bases to 50,000 bases in length, e.g., 1000 bases to 20,000 bases, or 5000 bases to 10,000 bases. The amount of DNA (e.g., human genomic DNA) used in a single mixture may be <10 ng, <3 ng, <1 ng, <0.3 ng, or <0.1 ng of DNA. In some approaches, the amount of DNA used in the single mixture may be less than 3,000×, e.g., less than 900×, less than 300×, less than 100×, or less than 30× of haploid DNA amount. In some approaches, the amount of DNA used in the single mixture may be at least 1× of haploid DNA, e.g., at least 2× or at least 10× haploid DNA amount.

Target nucleic acids may be isolated using conventional techniques, for example as disclosed in Sambrook and Russell, Molecular Cloning: A Laboratory Manual, cited supra. In some cases, particularly if small amounts of the nucleic acids are employed in a particular step, it is advantageous to provide carrier DNA, e.g., unrelated circular synthetic double-stranded DNA, to be mixed and used with the sample nucleic acids whenever only small amounts of sample nucleic acids are available, and there is danger of losses through nonspecific binding, e.g., to container walls and the like.

According to some embodiments of the invention, genomic DNA or other complex target nucleic acids are obtained from an individual cell or small number of cells with or without purification by any known method.

As described above, methods of the disclosure are useful for sequencing long nucleic acid fragments. Long fragments of genomic DNA can be isolated from a cell by any known method. A protocol for isolation of long genomic DNA fragments from human cells is described, for example, in Peters et al., Nature 487:190-195 (2012). In one embodiment, cells are lysed and the intact nuclei are pelleted with a gentle centrifugation step. The genomic DNA is then released through proteinase K and RNase digestion for several hours. The material can be treated to lower the concentration of remaining cellular waste, e.g., by dialysis for a period of time (i.e., from 2-16 hours) and/or dilution. Since such methods need not employ many disruptive processes (such as ethanol precipitation, centrifugation, and vortexing), the genomic nucleic acid remains largely intact, yielding a majority of fragments that have lengths in excess of 150 kilobases. In some approaches, the fragments are from about 5 to about 750 kilobases in length. In further embodiments, the fragments are from about 150 to about 600, about 200 to about 500, about 250 to about 400, and about 300 to about 350 kilobases in length. The smallest fragment that can be used for haplotyping is approximately 2-5 kb; there is no maximum theoretical size, although fragment length can be limited by shearing resulting from manipulation of the starting nucleic acid preparation.

In other embodiments, long DNA fragments are isolated and manipulated in a manner that minimizes shearing or absorption of the DNA to a vessel, including, for example, isolating cells in agarose in agarose gel plugs, or oil, or using specially coated tubes and plates.

According to another embodiment, in order to obtain uniform genome coverage in the case of samples with a small number of cells (e.g., 1, 2, 3, 4, 5, 10, 10, 15, 20, 30, 40, 50 or 100 cells from a microbiopsy or circulating tumor or fetal cells, for example), all long fragments obtained from the cells are barcoded using methods disclosed herein.

### 4.3 Barcode

According to one embodiment, a barcode-containing sequence is used that has two, three or more segments of which, one, for example, is the barcode sequence. For example, an introduced sequence may include one or more regions of known sequence and one or more regions of degenerate sequence that serves as the barcode(s) or tag(s). The known sequence (B) may include, for example, PCR primer binding sites, transposon ends, restriction endonuclease recognition sequences (e.g., sites for rare cutters, e.g., Not I, Sac II, Mlu I, BssH II, etc.), or other sequences. The degenerate sequence (N) that serves as the tag is long enough to provide a population of different-sequence tags that is equal to or, preferably, greater than the number of fragments of a target nucleic acid to be analyzed. The higher the N value, the less likely two molecules will share the same barcode.

According to one embodiment, the barcode-containing sequence comprises one region of known sequence of any selected length. According to another embodiment, the barcode-containing sequence comprises two regions of known sequence of a selected length that flank a region of degenerate sequence of a selected length, i.e., BₙNₙBₐ, where N may have any length sufficient for tagging long fragments of a target nucleic acid, including, without limitation, N=10, 11, 12, 13, 14, 15, 16, 17, 18, 19 or 20, and B may have any length that accommodates desired sequences such as transposon ends, primer binding sites, etc. For example, such an embodiment may be B₂₀N₁₅B₂₀.

In one embodiment, a two or three-segment design is utilized for the barcodes used to tag long fragments. This design allows for a wider range of possible barcodes by allowing combinatorial barcode segments to be generated by ligating different barcode segments together to form the full barcode segment or by using a segment as a reagent in oligonucleotide synthesis. This combinatorial design provides a larger repertoire of possible barcodes while reducing the number of full-size barcodes that need to be generated. In further embodiments, unique identification of each long fragment is achieved with 8-12 base pair (or longer) barcodes.

In one embodiment, two different barcode segments are used. A and B segments are easily modified to each contain a different half-barcode sequence to yield thousands of combinations. In a further embodiment, the barcode sequences are incorporated on the same adapter. This can be achieved by breaking the B adapter into two parts, each with a half barcode sequence separated by a common overlapping sequence used for ligation. The two tag components have 4-6 bases each. An 8-base (2×4 bases) tag set is capable of uniquely tagging 65,000 sequences. Both 2×5 base and 2×6 base tags may include use of degenerate bases to achieve optimal decoding efficiency.

In further embodiments, unique identification of each sequence is achieved with 8-12 base pair error correcting barcodes. Barcodes may have a length, for illustration and not limitation, of from 5-20 informative bases, usually 8-16 informative bases.

### 4.4 UMI

In various embodiments, unique molecular identifiers (UMIs) are used to distinguish individual DNA molecules from one another. The collection of adapters is generated, each having a UMI. Those adapters are attached to fragments or other source DNA molecules to be sequenced, and the individual sequenced molecules each has a UMI that helps distinguish it from all other fragments. In such implementations, a very large number of different UMIs (e.g., many thousands to millions) may be used to uniquely identify DNA fragments in a sample.

The UMI is at a length that is sufficient to ensure the uniqueness of each and every source DNA molecule. In some approaches, the unique molecular identifier is about 3-12 nucleotides in length, or 3-5 nucleotides in length. In some cases, each unique molecular identifier is about 3-12 nucleotides in length, or 3-5 nucleotides in length. Thus, a unique molecular identifier can be 3, 4, 5, 6, 7, 8, 9, 10, 11, 12, 13, 14, 15, 16, 17, 18, or more nucleotides in length.

A process of sequencing a target nucleic acid having repetitive regions can be carried out according to various schemes. In some embodiments the method involves co-barcoding the fragments to introduce barcodes occurs before mutagenesis; in other embodiments the method involves co-barcoding the fragments to introduce barcodes after mutagenesis. Described below are exemplary embodiments of the methods. A practitioner with skill in the arts of molecular biology and sequencing guided by this disclosure will recognize numerous variations of individual steps and reagents that can be incorporated into the schemes below.

### 5. Approach I (Mutagenesis after Co-Barcoding)

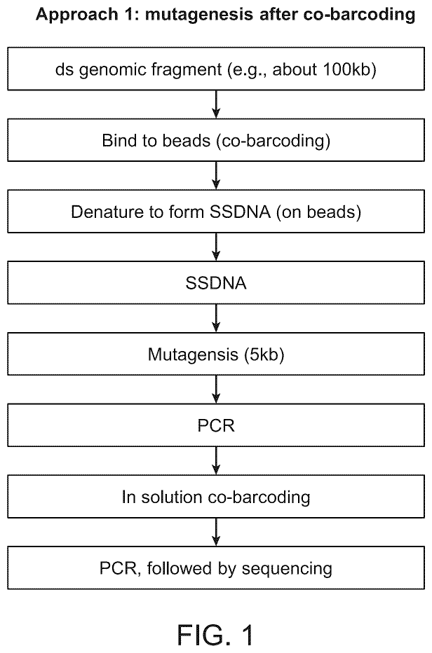

The general workflow of Approach I method is shown in FIG. 1. Briefly the method comprises co-barcoding the library of target nucleic acids on beads, thereby generating a plurality of barcoded target nucleic acid fragments for each target nucleic acid; denaturing the barcoded target nucleic acid fragments to form single-stranded barcoded fragments; subjecting the single-stranded barcoded fragments to mutagenesis while the single-stranded barcoded fragments remain attached to beads; amplifying the mutagenized single-stranded barcoded fragments to form mutagenized double-stranded barcoded fragments; in solution co-barcoding of each mutagenized double-stranded barcoded fragment form a plurality of second double-stranded barcoded fragments; sequencing the second double-stranded barcoded fragments; assembling the sequence reads from sequencing the second double-stranded barcoded fragments to obtain the sequence information of the mutagenized double-stranded barcoded fragments; and assembling the sequence information of the mutagenized double-stranded barcoded fragments to generate the sequence information of the target nucleic acid.

One exemplary approach is shown in FIG. 2A. Target nuclec acids (for example, long genomic DNA molecules) (201) with repeats (for example, CAAC) are bound to beads immobilized barcodes. Each long genomic DNA molecule is about 50 kb. stLFR is performed to insert copies of the barcode sequence in intervals, each interval is about 5-10 kb in length. Each of the barcoded fragments (202), having a length of 5-10 kb, is then denatured and mutagenized as described above to randomly convert A to G and C to T at low frequencies, thereby producing mutagenize barcoded fragments (203). In solution barcoding is performed to insert additional copies of same barcode sequence to the mutagenized barcoded fragments (3) and produce barcoded fragments (204), each has a barcode sequence and has a length that is suitable for sequencing (e.g., a length of 100-1000 bases, for example, 500 bases).

The barcoded fragments (203) are sequenced. Sequence reads sharing the same barcode sequence are assigned to the same long genomic DNA molecule (201). The original sequence of the long genomic DNA molecule (201) can be determined by performing base correction as disclosed in the section above entitled “assemble sequence information.” See FIG. 2B.

### 6. Approach II (Mutagenesis Before Co-Barcoding)

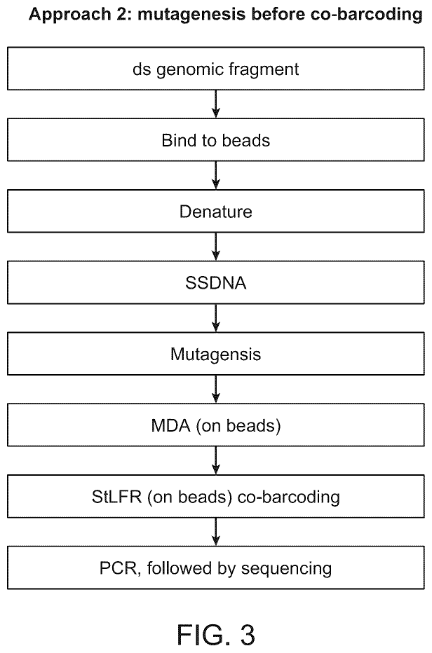

The general workflow of Approach II method is shown in FIG. 3. Briefly, the method comprises immobilizing the target nucleic acids on beads; denaturing the target nucleic acids to produce single-stranded target nucleic acids, wherein the single-stranded target nucleic acids remain attached on beads; subjecting the single-stranded target nucleic acids to mutagenesis while the single-stranded target nucleic acids; performing multiple displacement amplification (MDA) on beads to amplify the mutagenized single-stranded nucleic acids to form amplified mutagenized nucleic acids that remain attached on beads; co-barcoding the amplified mutagenized nucleic acids on the beads, thereby for each amplified mutagenized nucleic acid, producing a plurality of mutagenized barcoded fragments, each mutagenized barcoded fragment comprising the barcode sequence and a sequence portion of the amplified mutagenized nucleic acid, wherein at least some of the mutagenized barcoded fragment have different sequence portions of the amplified mutagenized nucleic acid; sequencing the mutagenized barcoded fragments to produce sequence reads; and assembling the sequence reads to produce sequence information of the amplified mutagenized nucleic acid molecules, thereby generating the sequence information of the target nucleic acid molecules.

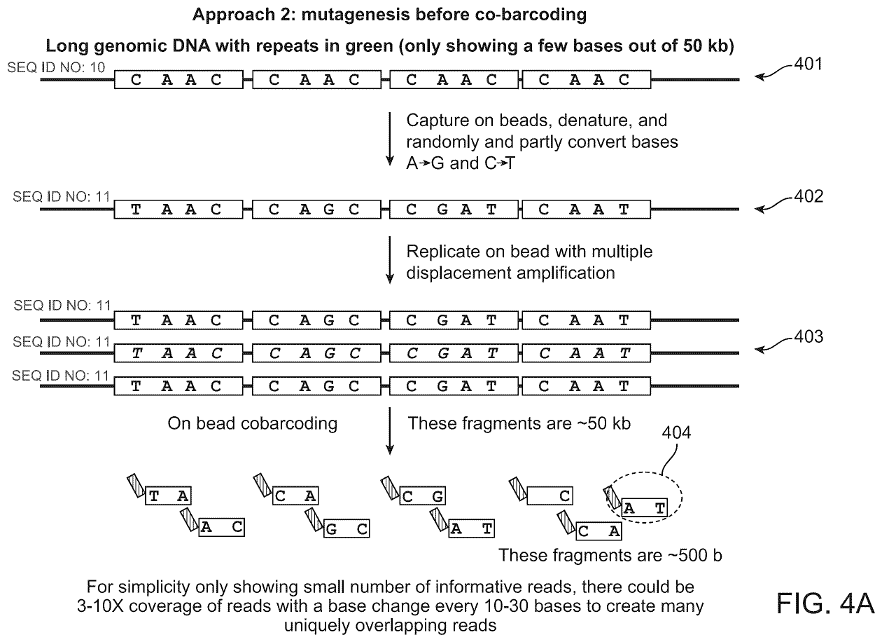

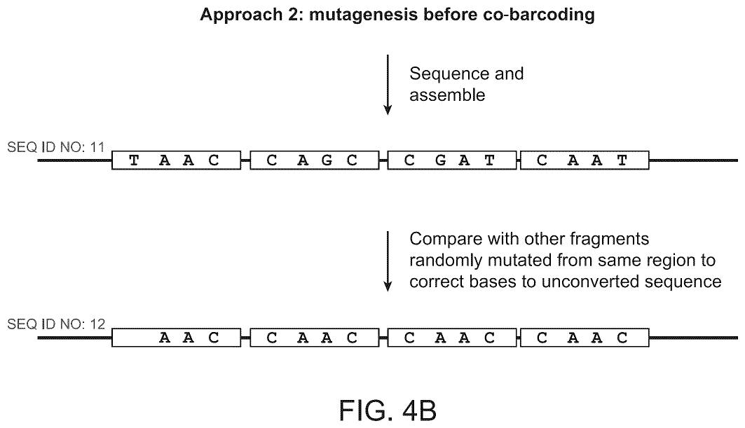

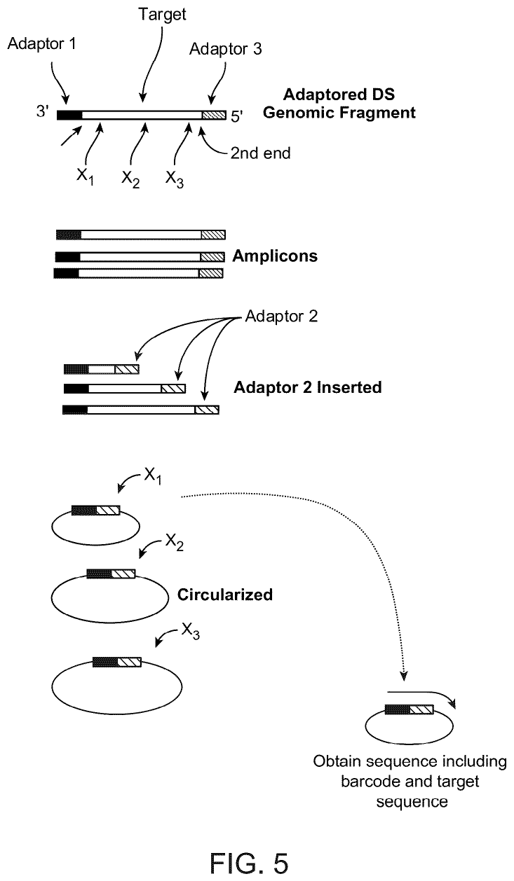

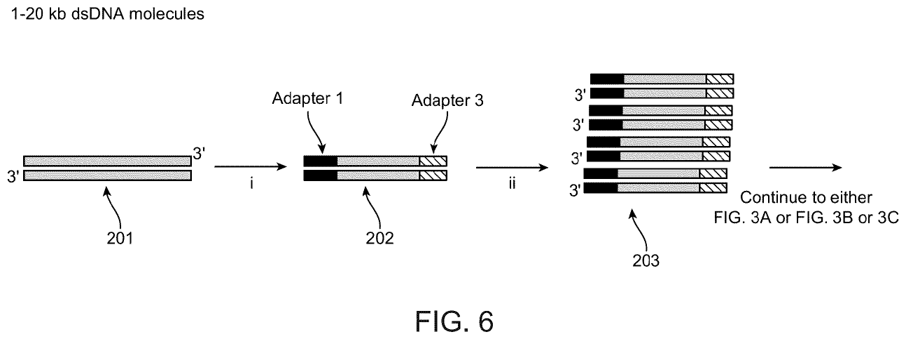

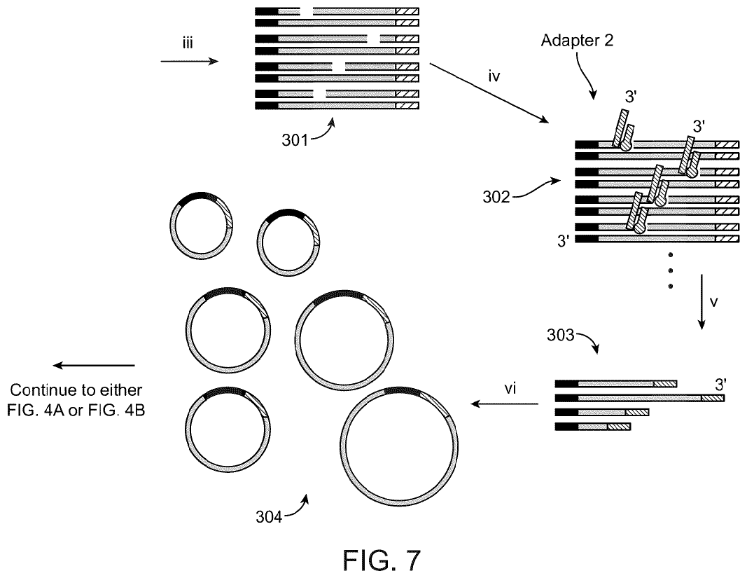

One exemplary approach is shown in FIG. 4. Briefly, long genomic DNA molecules (401) with repeats (for example, CAAC) are bound to beads. Each long genomic DNA molecule is about 50 kb. The captured genomic DNA molecules are denatured to form single-stranded DNA molecules. The single-stranded DNA molecules are then mutagenized as described above to randomly convert Ato G and C to T at low frequency. This results in mutagenized barcoded fragments (402). The mutagenized barcoded sequences (402) are amplified by multiple displacement amplification (MDA) on the beads, thus producing amplified mutagenized fragments (403). Co-barcoding on beads is performed to insert copies of a barcode sequence to the amplified mutagenized fragments (403) and produce barcoded fragments (404), each has the barcode sequence and has a length that is suitable for sequencing (e.g., a length of 100-1000 bases, for example, 500 bases).

### EXEMPLARY EMBODIMENTS

Embodiment 1 is a method of sequencing a library of target nucleic acid molecules, the method comprising: (a) mutagenizing and co-barcoding the library of target nucleic acid molecules, thereby producing a plurality of mutagenized barcoded fragments for each target nucleic acid molecule, wherein each of the plurality of mutagenized barcoded fragments comprises a barcode, where the barcode comprises a barcode sequence, wherein the plurality of mutagenized barcoded fragments produced from the same target nucleic acid share the same barcode sequence, wherein the mutagenized barcoded fragments produced from different target nucleic acid molecules have different barcode sequences, wherein the mutagenesis converts selected nucleic acid bases to different nucleic acid bases at a rate of 1% to 30%; (b) sequencing the mutagenized barcoded fragments to produce sequence reads; and (c) assembling the sequence reads to generate an assembled sequence of the target nucleic acid based on the barcode sequence in the sequence reads and mutation patterns.

Embodiment 2 is the method of Embodiment(s) 1, wherein the method further comprises deducing the unmutated sequence of each target nucleic acid by aligning the sequence reads and reverting mutated nucleotides in the sequence reads to unmutated nucleotides in silico.

Embodiment 3 is the method of Embodiment(s) 1 or 2, wherein the deducing the unmutated sequence of each target nucleic acid comprises comparing (1) the sequence information from the mutagenized barcoded fragments with (2) the sequence information of unmutagenized barcoded fragments of the target nucleic acid that was prepared in the same manner as the mutagenized barcoded fragments.

Embodiment 4 is the method of Embodiment(s) 1, wherein co-barcoding occurs before mutagenesis.

Embodiment 5 is the method of Embodiment(s) 4, wherein the method further comprises co-barcoding the plurality of mutagenized barcoded fragments in solution, thereby forming a plurality of second double-stranded barcoded fragments from each mutagenized barcoded fragment, wherein each second double-stranded barcoded fragment comprises the barcode and a sequence portion of the mutagenized barcoded fragment, and wherein at least some of the second double-stranded barcoded fragments comprise different sequence portions of the mutagenized barcoded fragment.

Embodiment 6 is the method of Embodiment(s) 1, wherein co-barcoding occurs after mutagenesis.

Embodiment 7 is the method of Embodiment(s) 1, wherein the co-barcoding is performed on beads.

Embodiment 8 is a method for sequencing a library of target nucleic acid molecules having repetitive regions, the method comprising co-barcoding the library of target nucleic acid molecules on beads, thereby generating a plurality of barcoded target nucleic acid fragments for each target nucleic acid molecule, wherein for each target nucleic acid molecule, each of the plurality of barcoded target nucleic acid fragments generated for the target nucleic acid comprises a barcode comprising a barcode sequence and a sequence portion of the target nucleic acid molecule, wherein at least some of the plurality of barcoded target nucleic acid fragments have different sequence portions of the target nucleic acid molecule, wherein the barcoded target nucleic acid fragments generated from the same target nucleic acid share identical barcode sequences, and the barcoded target nucleic acid fragments generated from different target nucleic acid molecules have different barcode sequences; wherein for the plurality of the barcoded target nucleic acid fragments generated for the target nucleic acid molecule, denaturing the barcoded target nucleic acid fragments to form single-stranded barcoded fragments, wherein the single-stranded barcoded fragments remain attached to the bead; subjecting the single-stranded barcoded fragments to mutagenesis, which mutates only selected nucleic acid bases at a rate of 1% to 30%, thus forming a group of mutagenized single-stranded barcoded fragments; amplifying the mutagenized single-stranded barcoded fragments to form mutagenized double-stranded barcoded fragments, for each mutagenized double-stranded barcoded fragment, co-barcoding the mutagenized double-stranded barcoded fragment in solution to form a plurality of second double-stranded barcoded fragments, each comprising a copy of the barcode sequence and a sequence portion of the mutagenized double-stranded barcoded fragment, and wherein at least some of the second double-stranded barcoded fragments have different sequence portions of the mutagenized double-stranded barcoded fragment, sequencing the second double-stranded barcoded fragments, and assembling the sequence reads from (ii) to obtain the sequence information of the mutagenized double-stranded barcoded fragments, and assembling the sequence information of the mutagenized double-stranded barcoded fragments to generate the sequence information of the target nucleic acid.

Embodiment 9 is the method of Embodiment(s) 8, wherein the second double-stranded barcoded fragments produced from each mutagenized double-stranded barcoded fragment further comprise positional barcodes comprising positional barcode sequences, wherein at least two positional barcode sequences are different, wherein the different positional barcode sequence denote different positions of the second double-stranded barcode fragments relative to the mutagenized double-stranded barcoded fragment.

Embodiment 10 is the method of Embodiment(s) 1, wherein the barcoded target nucleic acid fragments produced from each target nucleic acid have lengths of 1 kb-10 kb.

Embodiment 11 is a method for sequencing a library of target nucleic acid molecules comprising repetitive regions, the method comprising immobilizing the target nucleic acid molecules on beads, each bead is immobilized thereon a barcode comprising a barcode sequence that is unique to the bead, denaturing the target nucleic acid molecules to form single-stranded target nucleic acid molecules, wherein the single-stranded target nucleic acid molecules remain attached to beads, subjecting the single-stranded target nucleic acid molecules to mutagenesis, wherein the mutagenesis mutates only selected nucleic acid bases at a rate of 1% to 30%, thus forming a group of mutagenized single-stranded nucleic acid molecules, performing multiple displacement amplification (MDA) on beads to amplify the mutagenized single-stranded nucleic acid molecules to form amplified mutagenized nucleic acid molecules that remain attached to the beads, co-barcoding the amplified mutagenized nucleic acid molecules on the beads, thereby for each amplified mutagenized nucleic acid molecule, producing a plurality of mutagenized barcoded fragments, each mutagenized barcoded fragment comprising the barcode sequence and a sequence portion of the amplified mutagenized nucleic acid molecule, wherein at least some of the mutagenized barcoded fragment have different sequence portions of the amplified mutagenized nucleic acid molecule, sequencing the mutagenized barcoded fragments to generate sequence reads and assembling the sequence reads to produce sequence information of the amplified mutagenized nucleic acid molecule, thereby generating the sequence information of the target nucleic acid molecules.

Embodiment 12 is the method of Embodiment(s) 11, wherein intervals between adjacent barcodes in each co-barcoded first fragment has a length of between 15 bp-1500 bp.

Embodiment 13 is the method of any one of the claims above, wherein the co-barcoding fragments on beads comprises 1) introducing staggered single-stranded breaks into the target nucleic acid molecule, thereby producing a plurality of fragments, and 2) associating each fragment with a capture oligonucleotide, wherein the capture oligonucleotide comprises the barcode having a barcode sequence, wherein each of the plurality of fragments is associated with the same barcode sequence.

Embodiment 14 is the method of any one of the claims above, wherein the co-barcoding fragments on beads is by nick-ligation.

Embodiment 15 is the method of any one of the claims above, wherein the co-barcoding fragments on beads is by transposition.

Embodiment 16 is the method of any one of the claims above, wherein the mutations comprise one or more mutations selected from the group consisting of (i) C to T and (ii) A to G.

Embodiment 17 is the method of any one of Embodiment(s)s 1-16, wherein generating the sequence information of the target nucleic acid molecules comprises: assigning the two sequence reads having matching mutation patterns to the same region of the target nucleic acid molecule, or assigning two sequence reads having distinguishable mutation patterns to two different repetitive regions in the target nucleic acid.

While this invention has been disclosed with reference to specific aspects and embodiments, it is apparent that other embodiments and variations of this invention may be devised by others skilled in the art without departing from the true spirit and scope of the invention.

Each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date. As used below, any reference to a series of examples is to be understood as a reference to each of those examples disjunctively (e.g., “Examples 1-4” is to be understood as “Examples 1, 2, 3, or 4”).

### EXAMPLES

The following examples are offered for illustrative purposes and are not intended to limit the invention.

### 1. Example 1: Approach I (Mutagenesis after Co-Barcoding)

The method described below uses transposons, but the co-barcoding can also be performed using nicking-ligating stLFR, as disclosed in the international Application No. PCT/CN2022/107241, published as WO 2023/001262, the entire content of which is herein incorporated by reference.

### Transposons Preparation

To generate transposon adapters, 10 μL of 100 μM Transposon oligonucleotide (/5Phos/CGATCCTTGGTGATCATCGGACCTACGTCAGTGCTTGTCTTCCTAAGATGTGTATAAGAGACA G(SEQ ID NO: 1)),10 μL of 100 μM ME-U (/5Phos/CTGUCTCUTATACACAUCT(SEQ ID NO: 2)), and 10 μL of 3× annealing Buffer (0.03 M Tris-HCl pH 7.5 and 0.3 M NaCl) are combined in a PCR strip tube. For the gap ligation adapter, 10 μL of 100 μM GapTop (/5Phos/TCTGCTGAGTCGAGAACGTCT/3′ddC(SEQ ID NO: 3)), 10 μL of 100 μM GapBottom (CTCGACTCAGCAG/3′ddA(SEQ ID NO: 4)), and 10 μL of 3× Annealing Buffer are added and mixed in a separate PCR tube and incubatedat 70° C. for 3 minutes. The tube is then allowed to slowly cool to room temperature. The cooling step can be performed on a PCR thermocycler with a slow ramp setting of 0.1° C. per second. 9.6 μL of each hybridized transposon from the previous step are incubated with 23.53 μL of Tn5 (13.6 pmol/μL), and 46.87 μL of 1× Coupling Buffer (0.5× TE buffer and 50% glycerol) at 30° C. for 1 hour to generate the transposome complex. The coupled transposome complex can be used immediately or stored at −20° C. for up to 12 months. 40 ng of High Molecular Weight (HMW) DNA are transferred to a PCR strip tube and diluted with nuclease free water to a total volume of 32 μL. 2 μL of transposome prepared as described above are diluted with 60-300 μL TE buffer (10 mM Tris-HCl pH 8.0 and 0.1 mM EDTA) and mix by pipetting the mixture up and down 10 times. Different dilutions can be prepared to determine which provides the best size range for the specific application. In this step the transposons will be integrated into the genomic DNA. 10 μL of 5× Transposase Buffer (50 mM TAPS-NaOH pH 8.5, 25 mM MgCl₂, and 50% DMF) and 8 μL of diluted transposome are incubated with genomic DNA in the PCR tube. at 55° C. for 10 minutes. 20 μL of the product from previous step are transferred to a new PCR tube. The transposase are removed by adding 2 μL of 1% SDS and incubated at room temperature for 10 minutes. This will allow the genomic DNA to fragment enabling transposon incorporation to be viewed on an agarose gel. The product can then be loaded on a 0.5× TBE (50 mM Tris, 45 mM boric acid, and 0.5 mM EDTA) 1% agarose gel and run at 150 V for 40 minutes. Most of the transposon incorporated DNA will migrate in the range of 200 to 1,500 bp on the gel (FIG. 4). For MGI sequencing platforms, a smear with the center around 600 bp is ideal, this may vary depending on your application. Controls that were processed with the same steps but lack the transposon, the Tn5 enzyme, or genomic DNA can be loaded on the same gel. If the transposon incorporated DNA band is not the desired size, the usage of the coupling transposome should be adjusted until the smear is the desired size. If the size of the transposon integrated material looks correct, carefully pipette, using a wide bore tip, 1.25 μL of the remaining transposon inserted DNA product from to a 2 ml round bottom tube and diluting the mixture with 98.75 μL of TE buffer. Transfer 50 μL prepared stLFR beads (˜50 million), MGI Tech Co., Ltd., Catalog #940-000193-00) to a 1.5 mL microcentrifuge tube. The tube is placed on a magnetic rack for 2 minutes to collect beads to the side of the tube. The supernatant are discarded and the beads are resuspended in 100 μL of 2× Hybridization Buffer (0.1 M Tris-HCl pH 7.5, 0.2 M MgCl₂, and 0.1% Tween 20). 100 μL of resuspended beads are then transferred to the tube containing diluted DNA, mix gently by inverting the tube several times. The mixture of beads and DNA is then placed on a tube rotator set to the lowest speed setting in an oven at 60° C. and incubated for 10 minutes. The temperature of the oven is then lowered to 45° C., excess temperature can be removed by leaving the oven door open for several minutes. The mixture is then incubated on the slowest speed setting of the tube rotator for an additional 50 minutes. This step captures the transposon inserted DNA to the barcoded magnetic beads.

### Insertion of Transposon to DNA (Co-Barcoding on Beads)

Now the captured transposon inserted DNA can be connected to the barcoded adapter molecules on the beads through a ligation step. A ligation mix is prepared by mixing 52 μL of 5× Ligation Buffer (12.5% PEG-8000, 0.25 M Tris-HCl pH 7.8, 5 mM ATP, and 0.05 M DTT) and 8 μL of 2×10\hat{ }6 units/mL T4 DNA ligase. The tube containing the captured DNA on beads are taken out of the oven and allowed to cool down to room temperature. 60 μL of ligation mix is added to the tube containing the bead DNA mixture and inverted several times to mix. The tube is placed on tube rotator for 1 hour at the lowest speed setting at room temperature and then placed on the magnetic rack for 2 minutes to collect beads. The supernatant is then discarded and 180 μL of Low-Salt Wash Buffer (50 mM Tris-HCl pH 7.5, 150 mM NaCl, and 0.05% Tween 20) is added.

An adapter oligo digestion mix is then prepared to remove excess unused adapter. The mix is prepared by combining 10 μL of 10× TA Buffer (330 mM Tris-acetate pH 7.5, 660 mM potassium acetate, 100 mM magnesium acetate, and 5 mM DTT), 4.5 μL of 20,000 U/mL Exonuclease I, 1 μL of 100,000 U/mL Exonuclease III, and 74.5 μL of dH₂O. Place the tube on the magnetic rack for 2 minutes to collect beads. Discard the supernatant, remove the tube from the magnetic rack, and add the digestion mix from step 19 to the tube. Vortex lightly to resuspend beads and incubate on the tube rotator for 10 minutes at 37° C. on the lowest speed setting.

Denaturation to Form ssDNA

Add 11 μL of 1% SDS, vortex vigorously, and incubate for 10 minutes at room temperature on the tube rotator and the lowest speed setting. Briefly centrifuge the tube to collect all liquid and beads to the bottom of the tube. Place on the magnetic rack for 2 minutes to collect beads to the side of the tube. Discard supernatant and add 300 μL of Low-Salt Wash Buffer. Briefly vortex and centrifuge to resuspend beads and collect all liquid to the bottom of the tube. Place the tube on the magnetic rack for 2 minutes to collect beads to the side of the tube. Discard the supernatant and repeat this step one time. Prepare the Single-stranded binding protein (SSB) binding mix by combining 20 μL of Pre-Ligation Buffer (0.05 M Tris-HCl pH 7.5 and 0.02 M MgCl₂) and 4 μL of Tth SSB protein ((Novus Biologicals #NBP2-35314-1 mg) in a new 1.5 mL tube. Carefully remove any remaining supernatant from the beads making sure there is no Low-Salt Wash Buffer left in the tube. Add the SSB binding mix to the beads and vortex lightly to resuspend. Incubate on the tube rotator on the lowest speed setting for 30 minutes at 37° C.

Prepare the gap ligation mix by combining 48 μL of 3× Gap Ligation Buffer (30% PEG8000, 150 mM Tris-HCl pH 7.8, 30 mM MgCl₂, 3 mM ATP, 1.5 mM DTT, and 0.15 mg/mL BSA), 18 μL of the 16.7 μM gap ligation adapter prepared in step 1, and 10 μL of 2×10\hat{ }6 units/mL T4 DNA ligase. Add the ligation mix directly to the beads containing the SSB binding mix (the SSB binding mix is not removed from the beads prior to adding the ligation mix). Vortex lightly to resuspend beads and incubate on the tube rotator for 2 hours at room temperature on the slowest speed setting. Add 80 μL of Low-Salt Wash Buffer to the tube and place on the magnetic rack for 2 minutes to collect beads to the side of the tube. Discard the supernatant, remove the tube from the magnetic rack, and add 150 μL of Low-Salt Wash Buffer. Briefly vortex and centrifuge to resuspend beads and collect all liquid to the bottom of the tube. Place the tube on the magnetic rack for 2 minutes to collect beads to the side of the tube.

### Mutagenesis

Mutagenesis can be performed using standard bisulfite chemistry protocols and enzymatic protocols such as NEBNext® Enzymatic Methyl-seq Kit (E7120S) to convert cytosine bases to uracil bases. For these methods, we can titrate the amount of enzyme used for the base conversion so that a frequency of about 1-3% or 2-5% or 3-10% of all cytosines in a sample are converted. Additionally, 1 M sodium nitrate can be used to deaminate Adenine (A) and cytosine (C) as described by Mahdavi-Amiri et al. Chem. Sci. 2021 Jan. 14; 12 (2): 606-612.

Add 50 μL 0.1 M NaOH to beads. Incubate for 5 minutes at room temperature. Briefly vortex and centrifuge to resuspend beads and collect all liquid to the bottom of the tube. Place the tube on the magnetic rack for 2 minutes to collect beads to the side of the tube. Remove supernatant and wash beads once with 50 ul of 0.1 M NaOH. Add mix of 23.8 ul of dH₂O and 1.2 μL of acetic acid. Briefly vortex to resuspend beads. Place the tube on the magnetic rack for 2 minutes to collect beads to the side of the tube. Discard supernatant. Add mix of 23.8 ul of dH₂O and 1.2 μL of acetic acid. Briefly vortex to resuspend beads. Next add 25 μL of freshly prepared 2 M sodium nitrite (Sigma-Aldrich, 237213-5G). Mix thoroughly and incubate at room temperature on a rotator for 2-12 hours, depending on the amount of desired base conversion. Add 80 μL of Low-Salt Wash Buffer to the tube and place on the magnetic rack for 2 minutes to collect beads to the side of the tube. Discard the supernatant, remove the tube from the magnetic rack, and add 150 μL of Low-Salt Wash Buffer. Briefly vortex and centrifuge to resuspend beads and collect all liquid to the bottom of the tube. Place the tube on the magnetic rack for 2 minutes to collect beads to the side of the tube.

### Amplification

Prepare the PCR master mix by adding 75 μL of 2× Q5U mix (NEB M0597L), 0.75 μL of 100 μM PCR Primer 1 (TGTGAGCCAAGGAGTTG(SEQ ID NO: 5)), 0.75 μL of 100 μM PCR primer 2 (GCCTCCCTCGCGCCATCAG(SEQ ID NO: 6)), and 73.5 μL of dH₂O. Remove the wash buffer and add the PCR master mix to beads. Briefly vortex and centrifuge to collect all contents to the bottom of the tube (don't pellet beads). Divide the resuspended bead mix into two PCR tubes or two wells of a PCR plate (75 μL per well). Vortex lightly to resuspend beads just prior to placing the tubes or PCR plate on the thermocycler. PCR for 10 cycles.

Combine the two wells of PCR product into a single 1.5 mL tube. Place the tube on the magnetic rack for 2 minutes to collect beads to the side of the tube. Collect the supernatant and place in a new 1.5 mL tube. Add 150 μL of AMPure XP beads to purify PCR product. Allow the mixture to incubate at room temperature for 10 minutes. Place the tube on the magnetic rack for 2 minutes to collect beads to the side of the tube. Discard supernatant. Leave the tube on the magnetic rack and add 200 μL of freshly prepared 80% EtOH. Incubate at room temperature for 1 minute. Discard supernatant and repeat this step one time. Remove all remaining 80% EtOH. If necessary, briefly centrifuge tube to collect drops from the sides of the tube to the bottom of the tube. Resuspend AMPure XP beads in 75 μL of TE buffer by vortexing. Incubate at room temperature for 5 minutes. Place the tube on the magnetic rack for 2 minutes to collect beads to the side of the tube. Collect the supernatant and place in a new 1.5 mL tube. After purification the yield should be ˜50-400 ng of PCR product. DNA concentration can be measured using a Nanodrop, Qubit, or equivalent device. This purified product is now ready for the in solution cobarcoding strategies described in International Application, WO 2024/022207. The entire content of said application is herein incorporated by reference.

### 2. Example 2: Approach 2 (Mutagenesis Before Co-Barcoding)

Dilute genomic DNA from stock solution to 0.25 ng/uL in TE pH 8.0 buffer (10 mM Tris, 1 mM EDTA) for pre-binding to beads. Place 30 million barcoded beads on magnet to collect beads to the side of the tube. Remove and discard supernatant. Wash beads once with 200 ul of low salt wash buffer (LSWB, 0.05 M Tris-HCl pH 7.5, 0.15 M NaCl, and 0.05% Tween 20) and once with 200 ul of 1× HB (10% PEG-8000, 50 mM Tris HCl, pH8.3, 10 mM MgCl₂, 1 mM ATP, and 0.05 mg/mL BSA). Remove the tube containing the beads from the magnet and gently resuspend beads in 10 uL of 1× HB buffer. Mix 1 μL of 0.25 ng/ul genomic DNA with 9 μl of 1× HB buffer and this mix to the 30 million beads for a total volume of 20 ul. Gently mix several times by slowly pipetting with a wide bore pipette. Incubate at room temperature for 15 mins. to allow the genomic DNA bind to the beads.

### Mutagenesis

Mutagenesis can be performed using standard bisulfite chemistry protocols and enzymatic protocols such as NEBNext® Enzymatic Methyl-seq Kit (E7120S) to convert cytosine bases to uracil bases. For these methods it is necessary to titrate the amount of base conversion so that a frequency of about 1-3% or 2-5% or 3-10% of all cytosines in a sample are converted. Additionally, 1 M sodium nitrate can be used to deaminate Adenine (A) and cytosine (C) as described by Mahdavi-Amiri et al. Chem. Sci. 2021 Jan. 14; 12 (2): 606-612. This is our preferred method. Add 50 μL of denaturing buffer (0.2M KOH, 10 mM EDTA, and 10% PEG). Incubate for 5 minutes at room temperature. Briefly vortex and centrifuge to resuspend beads and collect all liquid to the bottom of the tube. Place the tube on the magnetic rack for 2 minutes to collect beads to the side of the tube. Discard supernatant. Add mix of 23.8 ul of dH₂O and 1.2 μL of acetic acid. Briefly vortex to resuspend beads. Place the tube on the magnetic rack for 2 minutes to collect beads to the side of the tube. Discard supernatant. Add mix of 23.8 ul of dH₂O and 1.2 μL of acetic acid. Briefly vortex to resuspend beads. Next add 25 μL of freshly prepared 2 M sodium nitrite (Sigma-Aldrich, 237213-5G). Mix thoroughly and incubate at room temperature on a rotator for 2-12 hrs. depending amount of desired base conversion. Add 5-10 ul of 1 mM protected 8mers (NNNNN*N*N*N, N represents any one of A, T, C, G and each*represent a phosphothiolate bond) and 5 ul of 2× denaturation buffer (0.4 M KOH, 20 mM EDTA, and 20% PEG). Samples were mixed and incubated at room temperature for 5 minutes.

### MDA

Add 15 uL of renaturing buffer (0.2M MOPS buffer, pH7.5 with 10% PEG). Gently mix. 70 uL of Phi29 mix (1× Phi29 buffer (NEB B0269SVIAL), 0.4 mM dNTPs, 0.02% Pluronic F-68 (Thermo Fisher 24040032), 5 U/mL of Pyrophosphatase (NEB M0361S), 0.4 U/uL Phi29 DNA polymerase (Qiagen P7020-HC-L), and 10% PEG) are added to the renatured mix from above. In addition, a 5-10 uM final concentration of the adapter hybridization oligo (G*TC*GT*CIGTGC*A*/ddC (SEQ ID NO: 7)) can be added. If needed, blocker oligos (TGTGAGCCAAGGAGTTGCTG(SEQ ID NO: 8) with a 3 prime inverted dT) spanning the known adapter sequence region can be added to protect from 8mer annealing and displacement of the adapter hybridization oligo during multiple displacement amplification. Gently mix and incubate at room temperature for 5 minutes. Place the tube on the magnetic rack for 2 minutes to collect beads to the side of the tube. Discard supernatant. Wash once with 200 uL of 1× HB buffer.

### Co-Barcoding on Beads

Immediately proceed to the nicking ligating stLFR process by adding 50 uL of reaction mix (0.06U/uL of Masterase (Qiagen EN31), 0.01U/uL of Exolll, 1 uM of L-oligo (GAGACGTTCTCGACTCAGCAGAN*N*N*N(4-7) (SEQ ID NO: 9) (N can vary from 4 to 7 bases and represents any one of A, T, C, G and each*represent a phosphothiolate bond, which are resistant to nucleases) and 12 U/uL of T4 DNA ligase in 1× HB buffer) to the beads. Gently mix and incubate at 10° C. for 30 seconds and 37° C. for 30 seconds for a total of 60 cycles. Resuspend beads periodically through the incubation by gently inverting the tube several times. Briefly vortex and centrifuge to resuspend beads and collect all liquid to the bottom of the tube. Place the tube on the magnetic rack for 2 minutes to collect beads to the side of the tube. Discard supernatant. Wash beads twice with 200 μL of LSWB.

### Amplification, Purification, and Sequencing

Prepare the PCR master mix by adding 75 μL of 2× Q5U mix (NEB M0597L), 0.75 μL of 100 μM PCR Primer 1 (TGTGAGCCAAGGAGTTG(SEQ ID NO: 5)), 0.75 μL of 100 μM PCR primer 2 (GCCTCCCTCGCGCCATCAG(SEQ ID NO: 6)), and 73.5 μL of dH₂O. Remove the wash buffer and add the PCR master mix to beads. Briefly vortex and centrifuge to collect all contents to the bottom of the tube (don't pellet beads). Divide the resuspended bead mix into two PCR tubes or two wells of a PCR plate (75 μL per well). Vortex lightly to resuspend beads just prior to placing the tubes or PCR plate on the thermocycler. PCR for 7 cycles.

Combine the two wells of PCR product into a single 1.5 mL tube. Place the tube on the magnetic rack for 2 minutes to collect beads to the side of the tube. Collect the supernatant and place in a new 1.5 mL tube. Add 150 μL of AMPure XP beads to purify PCR product. Allow the mixture to incubate at room temperature for 10 minutes. Place the tube on the magnetic rack for 2 minutes to collect beads to the side of the tube. Discard supernatant. Leave the tube on the magnetic rack and add 200 μL of freshly prepared 80% EtOH. Incubate at room temperature for 1 minute. Discard supernatant and repeat this step one time. Remove all remaining 80% EtOH. If necessary, briefly centrifuge tube to collect drops from the sides of the tube to the bottom of the tube.

Resuspend AMPure XP beads in 75 μL of TE buffer by vortexing. Incubate at room temperature for 5 minutes. Place the tube on the magnetic rack for 2 minutes to collect beads to the side of the tube. Collect the supernatant and place in a new 1.5 mL tube. After purification the yield should be ˜50-400 ng of PCR product. DNA concentration can be measured using a Nanodrop, Qubit, or equivalent device. This product is now ready to enter the sequencing process.

### 3. Example 3. Assembly of Sequences of after Mutagenesis and Sequencing

After sequencing, reads sharing the same barcode can be assumed to be from a single long DNA fragment. Mutated bases can then be used to find overlapping reads and assembly of the original long fragments can begin. In genomic regions of low complexity or where many repeats are in tandem, these mutated bases are helpful to determine the order and overlap of reads (see FIG. 6).

The mutagenesis process is random and incomplete. That means of the many long fragments covering a given region (approximately 10-100 if we assume a 50-100× whole genome sequence coverage and roughly 0.5-5× coverage of each long molecule) the overlap of mutated bases between these fragments will be low. This can help determine what the original (unmutated) base at each position is. Alternatively, some amount (10-30× whole genome coverage) of unmutated sequence data, either from the same process, but without mutagenesis, or from a standard sequencing library can be used to determine the unmutated base at each position.

What is claimed is:

1. A method of sequencing a library of target nucleic acid molecules, the method comprising:
(a) mutagenizing and co-barcoding the library of target nucleic acid molecules, thereby producing a plurality of mutagenized barcoded fragments for each target nucleic acid molecule,
wherein each of the plurality of mutagenized barcoded fragments comprises a barcode,
where the barcode comprises a barcode sequence,
wherein the plurality of mutagenized barcoded fragments produced from the same target nucleic acid share the same barcode sequence,
wherein the mutagenized barcoded fragments produced from different target nucleic acid molecules have different barcode sequences,
wherein the mutagenesis converts selected nucleic acid bases to different nucleic acid bases at a rate of 1% to 30%;
(b) sequencing the mutagenized barcoded fragments to produce sequence reads, thereby producing sequence reads, wherein at least some of the sequence reads are significantly overlapping; and
(c) assembling the sequence reads to generate an assembled sequence of the target nucleic acid based on the barcode sequence in the sequence reads and mutation patterns.

2. The method of claim 1, wherein the method further comprises deducing the sequence of each target nucleic acid by aligning the sequence reads and reverting mutated nucleotides in the sequence reads to unmutated nucleotides in silico.

3. The method of claim 1, wherein the deducing the sequence of each target nucleic acid comprises comparing (1) the sequence information from the mutagenized barcoded fragments with (2) the sequence information of unmutagenized barcoded fragments of the target nucleic acid that was prepared in the same manner as the mutagenized barcoded fragments.

4. The method of claim 1, wherein co-barcoding occurs before mutagenesis.

5. The method of claim 4, wherein the method further comprises co-barcoding the plurality of mutagenized barcoded fragments in solution, thereby forming a plurality of second double-stranded barcoded fragments from each mutagenized barcoded fragment,
wherein each second double-stranded barcoded fragment comprises the barcode and a sequence portion of the mutagenized barcoded fragment, and
wherein at least some of the second double-stranded barcoded fragments comprise different sequence portions of the mutagenized barcoded fragment.

6. The method of claim 1, wherein co-barcoding occurs after mutagenesis.

7. The method of claim 1, wherein the co-barcoding is performed on beads.

8. A method for sequencing a library of target nucleic acid molecules comprising repetitive regions, the method comprising
co-barcoding the library of target nucleic acid molecules on beads, thereby generating a plurality of barcoded target nucleic acid fragments for each target nucleic acid molecule,
wherein for each target nucleic acid molecule,
each of the plurality of barcoded target nucleic acid fragments generated for the target nucleic acid comprises a barcode comprising a barcode sequence and a sequence portion of the target nucleic acid molecule,
wherein at least some of the plurality of barcoded target nucleic acid fragments have different sequence portions of the target nucleic acid molecule,
wherein the barcoded target nucleic acid fragments generated from the same target nucleic acid share identical barcode sequences, and the barcoded target nucleic acid fragments generated from different target nucleic acid molecules have different barcode sequences;
wherein for the plurality of the barcoded target nucleic acid fragments generated for the target nucleic acid molecule, denaturing the barcoded target nucleic acid fragments to form single-stranded barcoded fragments, wherein the single-stranded barcoded fragments remain attached to the bead;
1) subjecting the single-stranded barcoded fragments to mutagenesis, which mutates only selected nucleic acid bases at a rate of 1% to 30%, thus forming a group of mutagenized single-stranded barcoded fragments;
2) amplifying the mutagenized single-stranded barcoded fragments to form mutagenized double-stranded barcoded fragments,
for each mutagenized double-stranded barcoded fragment,
(i) co-barcoding the mutagenized double-stranded barcoded fragment in solution to form a plurality of second double-stranded barcoded fragments, each comprising a copy of the barcode sequence and a sequence portion of the mutagenized double-stranded barcoded fragment, and
wherein at least some of the second double-stranded barcoded fragments have different sequence portions of the mutagenized double-stranded barcoded fragment,
(ii) sequencing the second double-stranded barcoded fragments, thereby producing a plurality of sequence reads, wherein at least some of the plurality of sequence reads are significantly overlapping, and
(iii) assembling the sequence reads from (ii) to obtain the sequence of the mutagenized double-stranded barcoded fragments, and
3) assembling the sequence of the mutagenized double-stranded barcoded fragments to generate the sequence of the target nucleic acid.

9. The method of claim 8, wherein the second double-stranded barcoded fragments produced from each mutagenized double-stranded barcoded fragment further comprise positional barcodes comprising positional barcode sequences,
wherein at least two positional barcode sequences are different, wherein the different positional barcode sequence denote different positions of the second double-stranded barcode fragments relative to the mutagenized double-stranded barcoded fragment.

10. The method of claim 1, wherein the barcoded target nucleic acid fragments produced from each target nucleic acid have lengths of 1 kb-10 kb.

11. A method for sequencing a library of target nucleic acid molecules comprising repetitive regions, the method comprising
immobilizing the target nucleic acid molecules on beads, each bead is immobilized thereon a barcode comprising a barcode sequence that is unique to the bead,
denaturing the target nucleic acid molecules to form single-stranded target nucleic acid molecules, wherein the single-stranded target nucleic acid molecules remain attached to beads,
subjecting the single-stranded target nucleic acid molecules to mutagenesis, wherein the mutagenesis mutates only selected nucleic acid bases at a rate of 1% to 30%, thus forming a group of mutagenized single-stranded nucleic acid molecules,
performing multiple displacement amplification (MDA) on beads to amplify the mutagenized single-stranded nucleic acid molecules to form amplified mutagenized nucleic acid molecules that remain attached to the beads,
co-barcoding the amplified mutagenized nucleic acid molecules on the beads, thereby for each amplified mutagenized nucleic acid molecule, producing a plurality of mutagenized barcoded fragments, each mutagenized barcoded fragment comprising the barcode sequence and a sequence portion of the amplified mutagenized nucleic acid molecule, wherein at least some of the mutagenized barcoded fragment have different sequence portions of the amplified mutagenized nucleic acid molecule,
sequencing the mutagenized barcoded fragments to generate sequence reads, thereby producing a plurality of sequence reads, wherein at least some of the plurality of sequence reads are significantly overlapping; and
assembling the sequence reads to produce sequence information of the amplified mutagenized nucleic acid molecule, thereby generating the sequence information of the target nucleic acid molecules.

12. The method of claim 11, wherein intervals between adjacent barcodes in each co-barcoded first fragment has a length of between 15 bp-1500 bp.

13. The method of claim 7, wherein the co-barcoding fragments on beads comprises
1) introducing staggered single-stranded breaks into the target nucleic acid molecule, thereby producing a plurality of fragments, and
2) associating each fragment with a capture oligonucleotide, wherein the capture oligonucleotide comprises the barcode having a barcode sequence, wherein each of the plurality of fragments is associated with the same barcode sequence.

14. The method of claim 7, wherein the co-barcoding fragments on beads is by nick-ligation.

15. The method of claim 7, wherein the co-barcoding fragments on beads is by transposition.

16. The method of claim 1, wherein the mutations comprise one or more mutations selected from the group consisting of (i) C to T and (ii) A to G.

17. The method of claim 1, wherein generating the sequence of the target nucleic acid molecules comprises: for two sequence reads that are significantly overlapping,
assigning the two sequence reads having matching mutation patterns to the same region of the target nucleic acid molecule, or
assigning two sequence reads having distinguishable mutation patterns to two different repetitive regionsin the target nucleic acid.


SEQUENCE LISTING (12 sequences)

SEQ ID NO: 1 - DNA, 64 bp, synthetic construct
  source 1..64: mol_type = other DNA
cgatccttggtgatcatcggacctacgtcagtgcttgtcttcctaagatgtgtataagagacag

SEQ ID NO: 2 - DNA, 19 bp, synthetic construct
  source 1..19: mol_type = other DNA
  modified_base order(4,8,17): mod_base = OTHER; note = Uracil
ctgtctcttatacacatct

SEQ ID NO: 3 - DNA, 22 bp, synthetic construct
  source 1..22: mol_type = other DNA
  modified_base 22: mod_base = OTHER; note = 2-Prime,3-Prime-dideoxycytosine
tctgctgagtcgagaacgtctc

SEQ ID NO: 4 - DNA, 14 bp, synthetic construct
  source 1..14: mol_type = other DNA
  modified_base 14: mod_base = OTHER; note = 2-Prime,3-Prime-dideoxyadenosine
ctcgactcagcaga

SEQ ID NO: 5 - DNA, 17 bp, synthetic construct
  source 1..17: mol_type = other DNA
tgtgagccaaggagttg

SEQ ID NO: 6 - DNA, 19 bp, synthetic construct
  source 1..19: mol_type = other DNA
gcctccctcgcgccatcag

SEQ ID NO: 7 - DNA, 13 bp, synthetic construct
  source 1..13: mol_type = other DNA
  modified_base 7: mod_base = i
  modified_base 1^2: mod_base = OTHER; note = Phosphothiolate bond
  modified_base 3^4: mod_base = OTHER; note = Phosphothiolate bond
  modified_base 5^6: mod_base = OTHER; note = Phosphothiolate bond
  modified_base 11..13: mod_base = OTHER; note = Phosphothiolate bond
  modified_base 13: mod_base = OTHER; note = 2-Prime,3-Prime-dideoxycytosine
gtcgtcngtgcac

SEQ ID NO: 8 - DNA, 20 bp, synthetic construct
  source 1..20: mol_type = other DNA
tgtgagccaaggagttgctg

SEQ ID NO: 9 - DNA, 29 bp, synthetic construct
  source 1..29: mol_type = other DNA
  modified_base 23..26: mod_base = OTHER; note = Phosphothiolate bond
  misc_feature 29: note = N can be repeated upto three more times.
gagacgttctcgactcagcagannnnnnn

SEQ ID NO: 10 - DNA, 16 bp, synthetic construct
  source 1..16: mol_type = other DNA
caaccaaccaaccaac

SEQ ID NO: 11 - DNA, 16 bp, synthetic construct
  source 1..16: mol_type = other DNA
taaccagccgatcaat

SEQ ID NO: 12 - DNA, 15 bp, synthetic construct
  source 1..15: mol_type = other DNA
aaccaaccaaccaac